SEQUENCE LISTING (3 sequences)

SEQ ID NO: 1 - DNA, 1680 bp, Human
atggcgacggcgaccatagctctccaggtcaatggccagcaaggaggggggtccgagccggcggcggcggcggcagtggtggcagcgggagacaaatggaaacctccacagggcacagactccatcaagatggagaacgggcagagcacagccgccaagctggggctgcctcccctgacgcccgagcagcaggaggcccttcagaaggccaagaagtacgccatggagcagagcatcaagagtgtgctggtgaagcagaccatcgcgcaccagcagcagcagctcaccaacctgcagatggcagcagtgacaatgggctttggagatcctctctcacctttgcaatcgatggcggctcagcggcagcgggcgctggccatcatgtgccgcgtctacgtgggctctatctactatgagctgggggaggacaccatccgccaggcctttgccccctttggccccatcaagagcatcgacatgtcctgggactccgtcaccatgaagcacaagggctttgccttcgtggagtatgaggtccccgaagctgcacagctggccttggagcagatgaactcggtgatgctggggggcaggaacatcaaggtgggcagacccagcaacatagggcaggcccagcccatcatagaccagttggctgaggaggcacgggccttcaaccgcatctacgtggcctctgtgcaccaggacctctcagacgatgacatcaagagcgtgtttgaggcctttggcaagatcaagtcctgcacactggcccgggaccccacaactggcaagcacaagggctacggcttcattgagtacgagaaggcccagtcgtcccaagatgctgtgtcttccatgaacctctttgacctgggtggccagtacttgcgggtgggcaaggctgtcacaccgcccatgcccctactcacaccagccacgcctggaggcctcccacctgccgctgctgtggcagctgctgcagccactgccaagatcacagctcaggaagcagtggccggagcagcggtgctgggtaccctgggcacacctggactggtgtccccagcactgaccctggcccagcccctgggcactttgccccaggctgtcatggctgcccaggcacctggagtcatcacaggtgtgaccccagcccgtcctcctatcccggtcaccatcccctcggtgggagtggtgaaccccatcctggccagccctccaacgctgggtctcctggagcccaagaaggagaaggaagaagaggagctgtttcccgagtcagagcggccagagatgctgagcgagcaggagcacatgagcatctcgggcagtagcgcccgacacatggtgatgcagaagctgctccgcaagcaggagtctacagtgatggttctgcgcaacatggtggaccccaaggacatcgatgatgacctggaaggggaggtgacagaggagtgtggcaagttcggggccgtgaaccgcgtcatcatctaccaagagaaacaaggcgaggaggaggatgcagaaatcattgtcaagatctttgtggagttttccatagcctctgagactcataaggccatccaggccctcaatggccgctggtttgctggccgcaaggtggtggctgaagtgtacgaccaggagcgttttgataacagtgacctctctgcgtga

SEQ ID NO: 2 - DNA, 687 bp, Human
atggagcgggtgaagatgatcaacgtgcagcgtctgctggaggctgccgagtttttggagcgccgggagcgagagtgtgaacatggctacgcctcttcattcccgtccatgccgagcccccgactgcagcattcaaagcccccacggaggttgagccgggcacagaaacacagcagcgggagcagcaacaccagcactgccaacagatctacacacaatgagctggaaaagaatcgacgagctcatctgcgcctttgtttagaacgcttaaaagttctgattccactaggaccagactgcacccggcacacaacacttggtttgctcaacaaagccaaagcacacatcaagaaacttgaagaagctgaaagaaaaagccagcaccagctcgagaatttggaacgagaacagagatttttaaagtggcgactggaacagctgcagggtcctcaggagatggaacgaatacgaatggacagcattggatcaactatttcttcagatcgttctgattcagagcgagaggagattgaagtggatgttgaaagcacagagttctcccatggagaagtggacaatataagtaccaccagcatcagtgacattgatgaccacagcagcctgccgagtattgggagtgacgagggttactccagtgccagtgtcaaactttcattcacttcatag

SEQ ID NO: 3 - DNA, 2124 bp, Human
atgaatcaggaactgctctctgtgggcagcaaaagacgacgaactggaggctctctgagaggtaacccttcctcaagccaggtagatgaagaacagatgaatcgtgtggtagaggaggaacagcaacagcaactcagacaacaagaggaggagcacactgcaaggaatggtgaagttgttggagtagaacctagacctggaggccaaaatgattcccagcaaggacagttggaagaaaacaataatagatttatttcggtagatgaggactcctcaggaaaccaagaagaacaagaggaagatgaagaacatgctggtgaacaagatgaggaggatgaggaggaggaggagatggaccaggagagtgacgattttgatcagtctgatgatagtagcagagaagatgaacatacacatactaacagtgtcacgaactccagtagtattgtggacctgcccgttcaccaactctcctccccattctatacaaaaacaacaaaaatgaaaagaaagttggaccatggttctgaggtccgctctttttctttgggaaagaaaccatgcaaagtctcagaatatacaagtaccactgggcttgtaccatgttcagcaacaccaacaacttttggggacctcagagcagccaatggccaagggcaacaacgacgccgaattacatctgtccagccacctacaggcctccaggaatggctaaaaatgtttcagagctggagtggaccagagaaattgcttgctttagatgaactcattgatagttgtgaaccaacacaagtaaaacatatgatgcaagtgatagaaccccagtttcaacgagacttcatttcattgctccctaaagagttggcactctatgtgctttcattcctggaacccaaagacctgctacaagcagctcagacatgtcgctactggagaattttggctgaagacaaccttctctggagagagaaatgcaaagaagaggggattgatgaaccattgcacatcaagagaagaaaagtaataaaaccaggtttcatacacagtccatggaaaagtgcatacatcagacagcacagaattgatactaactggaggcgaggagaactcaaatctcctaaggtgctgaaaggacatgatgatcatgtgatcacatgcttacagttttgtggtaaccgaatagttagtggttctgatgacaacactttaaaagtttggtcagcagtcacaggcaaatgtctgagaacattagtgggacatacaggtggagtatggtcatcacaaatgagagacaacatcatcattagtggatctacagatcggacactcaaagtgtggaatgcagagactggagaatgtatacacaccttatatgggcatacttccactgtgcgttgtatgcatcttcatgaaaaaagagttgttagcggttctcgagatgccactcttagggtttgggatattgagacaggccagtgtttacatgttttgatgggtcatgttgcagcagtccgctgtgttcaatatgatggcaggagggttgttagtggagcatatgattttatggtaaaggtgtgggatccagagactgaaacctgtctacacacgttgcaggggcatactaatagagtctattcattacagtttgatggtatccatgtggtgagtggatctcttgatacatcaatccgtgtttgggatgtggagacagggaattgcattcacacgttaacagggcaccagtcgttaacaagtggaatggaactcaaagacaatattcttgtctctgggaatgcagattctacagttaaaatctgggatatcaaaacaggacagtgtttacaaacattgcaaggtcccaacaagcatcagagtgctgtgacctgtttacagttcaacaagaactttgtaattaccagctcagatgatggaactgtaaaactatgggacttgaaaacgggtgaatttattcgaaacctagtcacattggagagtggggggagtgggggagttgtgtggcggatcagagcctcaaacacaaagctggtgtgtgcagttgggagtcggaatgggactgaagaaaccaagctgctggtgctggactttgatgtggacatgaagtga

(19) United States
(12) Patent Application Publication
(10) Pub. No.: US 2024/0100079 A1
(43) Pub. Date: Mar. 28, 2024

(54) METHODS AND COMPOSITIONS FOR REGENERATING HAIR CELLS IN THE INNER EAR OF ADULT MAMMALS

(71) Applicant: Massachusetts Eye and Ear Infirmary, Boston, MA (US)

(72) Inventors: Zheng-Yi Chen, Chestnut Hill, MA (US); Yi-Zhou Quan, Boston, MA (US)

(21) Appl. No.: 18/275,146

(22) PCT Filed: Feb. 2, 2022
(86) PCT No.: PCT/US2022/014952

§ 371 Date: Jul. 31, 2023

Related U.S. Application Data
(60) Provisional application No. 63/144,883, filed on Feb. 2, 2021.

(51) Int. Cl. A61K 31/713; A61K 9/00; A61K 31/166; A61K 31/18; A61K 31/19; A61K 31/352; A61K 33/14; A61P 27/16; C07K 14/47; C12N 15/86

(52) U.S. Cl. CPC A61K 31/713; A61K 9/0043; A61K 31/166; A61K 31/18; A61K 31/19; A61K 31/352; A61K 33/14; A61P 27/16; C07K 14/4702; C12N 15/86

(57) ABSTRACT

Provided herein are methods for regenerating hair cells in an adult mammalian inner ear using novel combinations of agents selected from the group consisting of a histone deacetylase (HDAC) inhibitor, one or more inhibitory nucleic acids targeting Fir, Mxi1, Fbxw7, or a combination thereof, a Wnt pathway activator, and a cAMP activator. The methods and compositions can be used to treat a subject with hearing loss or vestibular dysfunction.

### CLAIM OF PRIORITY

This application claims the benefit of U.S. Provisional Patent Application No. 63/144,883, filed on Feb. 2, 2021, which is incorporated by reference herein in its entirety.

### FEDERALLY SPONSORED RESEARCH OR DEVELOPMENT

This invention was made with government support under Grant Nos. R01DC006908 and UH3TR002636 awarded by the National Institutes of Health and Grant No. W81XWH1810331 awarded by the Department of Defense. The government has certain rights in the invention.

### FIELD OF THE INVENTION

The subject matter disclosed herein generally relates to methods for regenerating hair cells in the inner ear of adult mammalian, and more particularly to the use of a unique combination of agents that are amenable to use in clinical practice.

### BACKGROUND OF THE INVENTION

Hearing loss affects one in 500 newborns and half of the population over 70 years old, worldwide. Despite being the most common human sensory malfunction, there is currently no pharmacological therapy for hearing loss. Adult mammalian cochlear hair cells (HCs), responsible for converting sound signals into electrical impulses, totally lose the capacity to regenerate spontaneously after damage. Hair cell loss in adult cochleae is considered the major cause of hearing loss.

### SUMMARY OF THE INVENTION

The present disclosure is based, at least in part, on the discovery of a novel combination of small molecules and inhibitory nucleic acids that induce regeneration of hair cells in a transgenic mouse model of hearing loss. It was also demonstrated that the novel combination described herein can be used to regenerate hair cells in an adult wild-type mouse with chemically induced hair cell damage.

Accordingly, aspects of the present disclosure provide a method for reprogramming an adult mammalian inner ear for hair cell regeneration comprising contacting an adult mammalian inner ear with an effective amount of a histone deacetylase (HDAC) inhibitor and one or more inhibitory nucleic acids targeting Fir, Mxi1, Fbxw7, or a combination thereof, under conditions and for a time sufficient to produce a population of progenitor cells in the adult mammalian inner ear.

In some embodiments, the HDAC inhibitor is selected from the group consisting of Sodium Butyrate, Trichostatin A, hydroxamic acids, cyclic tetrapeptides, trapoxin B, depsipeptides, benzamides, electrophilic ketones, aliphatic acid compounds, pyroxamide, phenylbutyrate, valproic acid, hydroxamic acids, romidepsin, vorinostat (SAHA), belinostat (PXD101), LAQ824, panobinostat (LBH589), entinostat (MS275), CI-994 (N-acetyldinaline, also tacedinaline), Entinostat (SNDX-275; formerly MS-275), EVP-0334, SRT501, CUDC-101, JNJ-26481585, PCI24781, Givinostat (ITF2357), and mocetinostat (MGCD0103).

In some embodiments, the HDAC inhibitor is valproic acid, Trichosatin A, vorinostate (SAHA), or belinostat (PXD101).

In some embodiments, the one or more inhibitory nucleic acids is a small interfering RNA (siRNA), a short hairpin RNA (shRNA), or an antisense oligonucleotide. In some embodiments, the one or more inhibitory nucleic acids comprises inhibitory nucleic acids that target Fir and Mxi1.

In some embodiments, methods described herein further comprise contacting the mammalian cochlea with a Wnt agonist and/or a cAMP agonist. In some embodiments, the Wnt activator is lithium chloride (LiCl) and/or the cAMP activator is forskolin.

In some embodiments, the progenitor cells of the population express Six1, Eya1, Gata3, Sox2, Notch1, Hes5, or a combination thereof.

In some embodiments, the contacting occurs in the inner ear of a subject.

Aspects of the present disclosure provide a method for treating hearing loss or vestibular dysfunction in a subject comprising administering to an inner ear of a subject in need thereof an effective amount of a histone deacetylase (HDAC) inhibitor and one or more inhibitory nucleic acids targeting Fir, Mxi1, Fbxw7, or a combination thereof; and administering to the inner ear of the subject an effective amount of an Atoh1 activator.

In some embodiments, the HDAC inhibitor is selected from the group consisting of Sodium Butyrate, Trichostatin A, hydroxamic acids, cyclic tetrapeptides, trapoxin B, depsipeptides, benzamides, electrophilic ketones, aliphatic acid compounds, pyroxamide, phenylbutyrate, valproic acid, hydroxamic acids, romidepsin, vorinostat (SAHA), belinostat (PXD101), LAQ824, panobinostat (LBH589), entinostat (MS275), CI-994 (N-acetyldinaline, also tacedinaline), Entinostat (SNDX-275; formerly MS-275), EVP-0334, SRT501, CUDC-101, JNJ-26481585, PCI24781, Givinostat (ITF2357), and mocetinostat (MGCD0103).

In some embodiments, the HDAC inhibitor is valproic acid, Trichosatin A, vorinostate (SAHA), or belinostat (PXD101).

In some embodiments, the one or more inhibitory nucleic acids is a small interfering RNA (siRNA), a short hairpin RNA (shRNA), or an antisense oligonucleotide. In some embodiments, the one or more inhibitory nucleic acids targets Fir and Mxi1.

In some embodiments, the Atoh1 activator is a nucleic acid encoding Atoh1. In some embodiments, the nucleic acid encoding Atoh1 is comprised in a vector. In some embodiments, the vector is a viral vector. In some embodiments, the viral vector is selected from the group consisting of a retroviral vector, a lentiviral vector, an adenoviral vector, an adeno-associated viral vector, a herpes simplex viral vector, and a vaccinia viral vector.

In some embodiments, methods described herein further comprise administering to the subject a Wnt agonist and/or a cAMP agonist. In some embodiments, the Wnt activator is lithium chloride (LiCl) and/or the cAMP activator is forskolin.

In some embodiments, the subject is a human patient having noise-induced permanent deafness, drug-induced hearing loss, age-related hearing loss, sudden sensorineural hearing loss, hearing loss due to viral infection, tinnitus, vestibular dysfunction, or a combination thereof. In some embodiments, the subject is a human.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIGS. 1A-1C include data showing that VPA/siFM can reprogram sensory cells in the adult mouse cochlea. FIG. 1A: A schematic diagram illustrating the experimental procedure using VPA/siFM in WT mice, followed by ad-Atoh1-mcherry in vitro. FIG. 1B: VPA/siFM- or vehicle-(DMSO)-treated and ad-Atoh1-mcherry treated adult (P30) WT mice cochlea samples were labelled with MYO7A/mcherry. FIG. 1C: Quantification and comparison showed a significant increase of regenerated HCs in the cultured cochleae between VsiFsiM/ad-Atoh1 treated or Vehicle/ad-Atoh1 treated groups. *p<0.05, Student's t-test. Error bar, mean±SEM; n=5 for each group. SE: Sensory region; Lib: Limbus region. V: VPA; siF: siFIR; siM: siMxi1. N=5 in each group. Scale bars: 10 m.

FIGS. 2A-2C include schematic diagrams illustrating the various combinations of small molecules and siRNAs that failed to reprogram and regenerate hair cells in cultured adult mouse cochlea.

FIGS. 3A-3D include data showing that the cocktail (VPA/siFIR/siMxi1) treatment regenerates HCs in the cultured adult mouse utricle after neomycin damage. FIG. 3A: A schematic diagram illustrating the experimental procedure of utricle samples treated with neomycin. FIG. 3B: Neomycin treated adult wild-type mouse utricle samples showed loss of hair cells (stained with MYO7A) compared to untreated utricle samples. FIG. 3C: A schematic diagram illustrating the experimental procedure of utricle samples treated with neomycin and the cocktail (VPA/siFIR/siMxi1). FIG. 3D: After neomycin treatment for 5 days, cultured utricles subsequently treated with VPA/siFIR/siMxi1 showed an increase in hair cells (MYO7A positive) than vehicle (DMSO) treated utricles. SOX2 labels both hair cells and supporting cells.

FIGS. 4A-4B include data showing the reprogramming effect of the cocktail in vitro. FIG. 4A: A schematic diagram illustrating the experimental procedure of cochlea samples treated by the combination of small molecules (VLFsiFsiM). FIG. 4B: qRT-PCR in cultured adult WT mouse cochlea after 4-day cocktail (VLFsiFsiM) treatment. A selected group of inner ear progenitor genes were up-regulated including Six1, Eya1, Gata3 and Sox2, Notch1 and its target Hes5. Some inner ear progenitor genes (Hes1, Foxg1 and Dlx5) showed little expression level change. Stem cell genes (Fut4, Nanog and Alpl) or differentiation genes (Prox1, P27^{kip1} or Cdknlb) were not up-regulated. *p<0.05, **p<0.01, ****p<0.0001, two-tailed unpaired Student's t-test. Error bar, mean±SEM, n=3. n is the number of biologically independent samples. V: VPA; L:LiCl; F:FSK; siF: siFIR; siM: siMxi1.

FIGS. 5A-5L include data showing that the small molecule and siRNA cocktail (VLFsiFsiM) induced robust hair cell regeneration. FIG. 5A: A schematic diagram illustrating the experimental procedure of cochlea samples treated by the application of a combination of small molecules and siRNA (VLFsiFsiM), and HC induction by ad-Atoh1 in the Atoh1-GFP mice in vitro. FIGS. 5B-5C: VLFsiFsiM (or Vehicle DMSO)/ad-Atoh1 treated adult (P30) Atoh1-GFP mice cochlea samples were labeled with MYO7A (dark gray)/Atoh1-GFP (light gray) in the sensory epithelial region. FIGS. 5D-5G: Quantification and comparison showed an increase of regenerated HCs (GFP+MYO7A labeling) in the sensory epithelium (SE) or the limbus region (Lib) of cultured cochleae in DLFsiFsiM/ad-Atoh1 treated samples compared to Vehicle/ad-Atoh1 treated samples. The number of infected cells (GFP positive) were the same between the two groups. FIGS. 5H-5I: VLFsiFsiM/ad-Atoh1 treated adult (P30) Atoh1-GFP mice cochlea samples were labeled with Atoh1-GFP/FM1-43/MYO7A, an indication that the hair cells were functional. FIG. 5J: A schematic diagram illustrating the experimental procedure of lineage tracing by Tamo induction of Sox2CreER/tdT plus VLFsiFsiM, and HC induction by ad-Atoh1 in vitro. FIG. 5K: Tamo/VLFsiFsiM/ad-Atoh1 treated adult (P30) Sox2CreER/tdT mice cochlea samples were labelled with MYO7A/Sox2-tdT. FIG. 5L: Enlarged images from (FIG. 5K). Arrow indicates MYO7A/Sox2-tdT double positive cells. Arrowhead indicates MYO7A+/Sox2-tdT-cells, demonstrating that the regenerated hair cells were from transdifferentiation of supporting cells. T, Tamo: 4-hydroxytamoxifen. V:VPA; L:LiCl; F:FSK; siF:siFIR; siM:siMxi1. **p<0.01, ****p<0.0001, Student's t-test. Error bar, mean±SEM; N=5-6 in each group. Scale bars: 10 m.

FIG. 6 includes data showing that various HDAC inhibitors in combination with Myc modulator siRNAs can be used to efficiently regenerate hair cells in adult cochlea in vitro after Ad-Atoh1 infection. L:LiCl; F:FSK; siF:siFIR; siM:siMxi1.

FIGS. 7A-7C include data showing that siRNA for the Myc modulator Fbxw7 alone or in combination with siRNA for the Myc modulators Fir and Mxi1 and the HDAC inhibitor VPA promotes hair cell regeneration with different efficiency in adult wild-type cochlea in vitro.

FIGS. 8A-8C include data from the severe HC loss model in vivo. FIG. 8A: A schematic diagram illustrating the experimental procedure of C57BL/6j mice treated by Kanamycin/Furosemide in vivo. FIG. 8B: Kana/Furo treated adult wild-type mouse cochlea samples stained with MYO7A/SOX2. FIG. 8C: Quantification and comparison of hair cells and supporting cells (HCs/SCs) between the freshly dissected and Kana/Furo treated ears. The data showed that after Kanamycin/Furosemide treatment, most outer hair cells were killed across the entire cochlea turns while the supporting cells were preserved. ****p<0.0001, two-tailed unpaired Student's t-test. Error bar, mean±SEM; N=5 in each group. Source data are provided as a Source Data file. Scale bars: 10 m.

FIGS. 9A-9C include data showing robust regeneration of new HCs in the severe HC loss model in vivo. FIG. 9A: A schematic diagram illustrating the experimental procedure of the C57BL/6j mice treated by Kanamycin/Furosemide, a combination of small molecules (or Vehicle), and HC induction by ad-Atoh1-mCherry in vivo. FIG. 9B: Kana/Furo/VLFsiFsiM (or Vehicles)/ad-Atoh1-mCherry treated adult wild-type mice cochlea samples labeled with ESPN/mCherry. In control group without the cocktail (VLFsiFsiM) treatment, few remaining outer hair cells were seen. In contrast, after the cocktail treatment, robust hair cell regeneration was seen across the cochlear turns. FIG. 9C: Quantification and comparison showed, in the outer hair cell (OHC) region, a significant increase in the number of hair cells in the cocktail treated inner ear compared to vehicle treated ears across the cochlear turns. In the inner hair cell (IHC) region, due to the lack of ad-Atoh1-mCherry infection and the survival of IHC, the IHC number is not changed. D:Dox; L:LiCl; F:FSK; siF:siFIR; siM:siMxi1. **p<0.01, ***p<0.001, ****p<0.0001, two-tailed unpaired Student's t-test. Error bar, mean±SEM; N=5-6 in each group. Source data are provided as a Source Data file. Scale bars: 10 m.

FIGS. 10A-10B include data showing low magnification of the new HCs in vivo. FIG. 10A: A schematic diagram illustrating the experimental procedure of the C57BL/6j mice treated by Kanamycin/Furosemide, a combination of small molecules (or Vehicle), and HC induction by ad-Atoh1-mCherry in vivo. FIG. 10B: Kana/Furo/VLFsiFsiM (or Vehicles)/ad-Atoh1-mCherry treated adult wild-type mice cochlea samples from apex to apex-mid labeled with ESPN/mCherry. D:Dox; L:LiCl; F:FSK; siF:siFIR; siM:siMxi1. Scale bars: 20 μm.

FIGS. 11A-11C include data showing robust regeneration of new HCs due to supporting cell transdifferentiation in the severe HC loss model in vivo. FIG. 11A: A schematic diagram illustrating the experimental procedure of the C57BL/6j mice treated by Kanamycin/Furosemide, a combination of small molecules (or Vehicles), and HC induction by ad-Atoh1-mCherry in vivo. FIG. 11B: Kana/Furo/VLFsiFsiM/ad-Atoh1-mCherry treated adult wild-type mouse cochlea samples stained with ESPN/SOX2/mCherry (arrowheads). Double-labeling of ESP-SOX2 is an indication that the hair cells (MYO7A) are derived from transdifferentiation of supporting cells (SOX2). FIG. 11C: Quantification and comparison showed a decrease in non-transdifferentiated SCs (ESPN−/SOX2+) after the cocktail treatment as the result of transdifferentiation of supporting cells to hair cells, compared to the freshly dissected ears. ****p<0.0001, two-tailed unpaired Student's t-test. Error bar, mean SEM; N=5 in each group. Scale bars: 20 m.

The details of one or more embodiments of the invention are set forth in the description below. Other features or advantages of the present invention will be apparent from the following drawings and detailed description of several embodiments, and also from the appended claims.

### DETAILED DESCRIPTION

Hair cell loss is the major cause of permanent hearing loss in humans. Strategies to overcome irreversible cochlear hair cell damage and loss in mammals are of vital importance to hearing recovery. In mature mammalian cochleae, co-activation of c-Myc and Notch1 reprograms the supporting cells and promotes hair cell regeneration. Given the crucial role of Myc in cell cycle, cell growth and stem cells, modulation of Myc by a clinically relevant approach is a priority in hair cell regeneration in the inner ear. We have shown that Myc plays an essential in hair cell regeneration. However, it is difficult to activate Myc using small molecules and MYC protein is unstable and difficult to produce. We have therefore focused on the identification of methods for activating Myc to a sufficient level for hair cell regeneration. We have additionally screened for multiple targets including HDAC inhibitors and inner ear development pathways to identify those that work with Myc to regenerate hair cells efficiently in vitro and in vivo.

As described herein, combinations of small molecules and inhibitory nucleic acids were used to regenerate hair cells in adult wild-type mice. Supporting cell origin of regenerated hair cells was confirmed by lineage tracing. Regenerated hair cells were labeled with multiple mature hair cell markers including SLC26A5 (prestin) for outer hair cells and SLC17A8 (VGLUT3) for inner hair cells. The new hair cells formed connections with adult spiral ganglion neurons and took up FM1-43 dye, an indication of the presence of the transduction complex. Additionally, efficient hair cell regeneration in a severe hair cell loss mouse model in vivo was demonstrated. In sum, the experimental results described herein identified a combinatorial approach that uses small molecules and inhibitory nucleic acids to regenerate hair cells in wild-type mature mammalian cochleae, laying the foundation for hearing restoration with a clinically relevant approach.

To restore hearing function, strategies for HCs regeneration are actively being pursued. Various lines of evidence suggest that HC regeneration in mammals is possible. Spontaneous HC regeneration occurs in lower vertebrates like birds and fish. Embryonic and neonatal mouse cochleae also retain the capacity to regenerate HCs by enhancing expression of specific genes essential for HC development. Hair cell regeneration in neonatal rodents was achieved by altering different signal pathways. Activating Sonic Hedgehog signaling resulted in both cell cycle re-entry in cochlear sensory epithelia and HC regeneration. The ERBB2 pathway was also involved in promoting supporting cell proliferation and increased MYO7A+ cell generation in neonatal mice.¹² However, this capacity to generate new HCs decreases rapidly 2 weeks after birth, even after altering expression of HC regenerating signal pathways⁹, and none of the treatments above were sufficient to regenerate cochlear HCs in adult mice.

It was previously shown that, by transient co-activation of Myc and NICD (Notch1 intracellular domain), the adult mouse cochlea can be successfully reprogrammed to a relatively younger stage and regain progenitor capacity, with generation of many new HCs following Atoh1 overexpression both in vitro and in vivo.¹⁶ Nevertheless, this “achievement” only occurs in transgenic animals, which is not applicable for clinical study in human patients. Thus, HCs regeneration in human cochleae remains particularly challenging.

By screening various combinations of small molecules and inhibitory nucleic acids as described herein, a unique combination of agents were identified that could modulate Myc and reprogram terminally differentiated cochlear epithelial cells, including Sox2+ supporting cells (SCs), to regain progenitor capacity and robustly regenerate new HCs in adulthood. Moreover, by comparing newly regenerated HCs of the different HC loss mouse models, it was revealed that the different reprogramming approaches in the adult cochlea led to different maturation levels of new HCs. Thus, the experimental data provided herein demonstrates that robust regeneration of cochlear HCs can be achieved in adult mice using a clinically relevant approach.

Accordingly, the present disclosure provides, in some aspects, methods for regenerating hair cells in adult wild-type cochlea and utricle using a unique combination of agents.

### I. Agents for Regenerating Hair Cells (HCs) in Adult Mammalian Cochlea

Provided herein are methods for regenerating hair cells in adult mammalian cochlea that involve (1) a reprogramming step in which the adult mammalian cochlea can be successfully reprogrammed to a relatively younger stage, thereby regaining progenitor capacity; and (2) a transdifferentiation step in which activation of HC fate-determining factor (Atoh1) in the reprogrammed adult progenitors leads to HC regeneration.

In the reprogramming step described herein, mammalian cochlea are reprogrammed to a less differentiated state using a unique combination of agents that includes a HDAC inhibitor and one or more inhibitory nucleic acids that target Fir, Mxi1, Fbxw7, or a combination thereof. In some embodiments, the unique combination of agents for use in methods described herein also include a Wnt agonist and/or a cAMP agonist.

As used herein, an “agent” refers to any molecule (e.g., a small molecule, a nucleic acid) capable of reprogramming mammalian cochlea for hair cell regeneration and/or regenerating hair cells in the mammalian cochlea. Agents for use in methods described include an HDAC inhibitor, an inhibitory nucleic acid targeting Fir, Mxi1, Fbxw7, or a combination thereof, a Wnt agonist, a cAMP agonist, and an Atoh1 activator.

(a) Histone Deacetylase (HDAC) Inhibitors

Methods described herein involve use of an HDAC inhibitor for reprogramming adult mammalian cochlea to a less differentiated, progenitor state. In some embodiments, the HDAC inhibitor is an HDAC class I inhibitor, an HDAC class II inhibitor, an HDAC class III inhibitor, and/or a pan-HDAC inhibitor. In some embodiments, the HDAC class III inhibitor is a SIRT1 inhibitor and/or a SIRT2 inhibitor.

As used herein, a “HDAC inhibitor” refers to a compound that binds and inhibits one or more HDACs, thereby affecting the enzyme activity of the HDAC. An HDAC refers to any one of a family of enzymes that catalyze the removal of acetyl groups from the F-amino groups of lysine residues at the N-terminus of a histone. Unless otherwise indicated by context, the term “histone” is meant to refer to any histone protein, including HI, H2A, H2B, H3, H4, and H5, from any species. Human HDAC proteins are separated into four classes: class I includes HDAC1, HDAC2, HDAC3, and HDAC8; class II includes HDAC4, HDAC5, HDAC7, and HDAC9; class III includes SIRT1, SIRT2, SIRT3, SIRT4, SIRT5, SIRT6, and SIRT7; and class IV includes HDAC11. An HDAC inhibitor may be a pan-HDAC inhibitor or exhibit selectivity towards one or more HDACs.

In some embodiments, the HDAC inhibitor is Sodium Butyrate, Trichostatin A, hydroxamic acids, cyclic tetrapeptides, trapoxin B, depsipeptides, benzamides, electrophilic ketones, aliphatic acid compounds, pyroxamide, phenylbutyrate, valproic acid, hydroxamic acids, romidepsin, vorinostat (SAHA), belinostat (PXD101), LAQ824, panobinostat (LBH589), entinostat (MS275), CI-994 (N-acetyldinaline, also tacedinaline), Entinostat (SNDX-275; formerly MS-275), EVP-0334, SRT501, CUDC-101, JNJ-26481585, PCI24781, Givinostat (ITF2357), mocetinostat (MGCD0103), or a combination thereof. Examples of HDAC inhibitors include, but are not limited to, compounds listed in Table 1.

TABLE 1
Exemplary HDAC inhibitors.

| Compound | Target |
|---|---|
| Vorinostat | HDAC class I (HDAC1, 2, 3, and 8) |
| (rINN; suberanilohydroxamic acid; suberoylanilide hydroxamic | and HDAC class II (IIa: HDAC4, 5, |
| acid; SAHA (suberoyl + anilide + hydroxamic acid abbreviated); N- | 7, and 9; IIb: 6 and 10) |
| Hydroxy-N′-phenyloctanediamide; Zolinza ®) | |
| Trichostatin A | HDAC class I (HDAC1, 2, 3, and 8) |
| (TSA; (2E,4E,6R)-7-(4-(Dimethylamino)phenyl)-N-hydroxy-4,6- | and HDAC class II (IIa: HDAC4, 5, |
| dimethyl-7-oxo-2,4-heptadienamide) | 7, and 9; IIb: 6 and 10) |
| belinostat | HDAC |
| (PXD101; Beleodaq) | |
| Valproic acid | HDAC |
| (VPA; sodium valproate; Sodium 2-propylpentanoate) | |
| FK 228 | HDAC class I |
| (Depsipeptide; FR 901228; Romidepsin; Cyclo[(2Z)-2-amino-2- | (HDAC1, 2, 3, and 8), |
| butenoyl-L-valyl-(3S,4E)-3-hydroxy-7-mercapto-4-heptenoyl-D- | HDAC4, and HDAC6 |
| valyl-D-cysteinyl], cyclic (3-5) disulfide) | |
| Sodium butyrate | HDAC |
| (Butanoic acid sodium salt; NaB) | |
| LMK 235 | HDAC4 and HDAC5 |
| (N-[[6-(Hydroxyamino)-6-oxohexyl]oxy]-3,5-dimethylbenzamide) | |
| Scriptaid | HDAC |
| (N-Hydroxy-1,3-dioxo-1H-benz[de]isoquinoline-2(3H)- | |
| hexanamide) | |
| M 344 | HDAC |
| (4-(Diethylamino)-N-[7-(hydroxyamino)-7-oxoheptyl]benzamide) | |
| SBHA | HDAC1 and HDAC3 |
| (N,N′-Dihydroxyoctanediamide; suberic bishydroxamate) | |
| CBHA | HDAC1 and HDAC3 |
| (m-carboxycinnamic acid bishydroxamide) | |
| HMBA | HDAC |
| (hexamethylene bisacetamide). | |
| Tubacin | HDAC6 |
| (N-[4-[(2R,4R,6S)-4-[[(4,5-Diphenyl-2-oxazolyl)thio]methyl]-6-[4- | |
| (hydroxymethyl)phenyl]-1,3-dioxan-2-yl]phenyl]-N′- | |
| hydroxyoctanediamide) | |
| Sodium 4-phenylbutyrate | HDAC |
| (4-PB; sodium pheylbutyrate; 4-Phenylbutyric acid, sodium salt; 4- | |
| phenylbutyrate) | |
| MC 1568 | HDAC class IIa |
| (3-[5-(3-(3-Fluorophenyl)-3-oxopropen-1-yl)-1-methyl-1H-pyrrol- | (HDAC4, 5, 7, and 9) |
| 2-yl]-N-hydroxy-2-propenamide) | |
| Compound 9 | HDAC class IIa |
| (Mai et al., J. Med. Chem., 2005; 48: 3344) | (HDAC4, 5, 7, and 9) |
| Compound 24 | HDAC class IIa |
| (Mai et al., J. Med. Chem., 2005; 48: 3344) | (HDAC4, 5, 7, and 9) |
| TC-H 106 | HDAC class I |
| (N1-(2-Aminophenyl)-N7-(4-methylphenyl)heptanediamide; | (HDAC1, 2, 3, and 8) |
| Pimelic Diphenylamide 106) | |
| Pyroxamide | HDAC1 |
| (N-Hydroxy-N′-3-pyridinyloctanediamide) | |
| NCH 51 | HDAC |
| (PTACH; 2-Methylpropanethioic acid S-[7-oxo-7-[(4-phenyl-2- | |
| thiazolyl)amino]heptyl] ester) | |
| NCH 31 | HDAC |
| PCI 34051 | HDAC8 |
| (N-Hydroxy-1-[(4-methoxyphenyl)methyl]-1H-indole-6- | |
| carboxamide) | |
| thiophene benzamide | HDAC1 and HDAC2 |
| KD 5170 | HDAC class I (HDAC1, 2, 3, and 8) |
| (S-[2-[6-[[[4-[3-(Dimethylamino)propoxy] phenyl]sulfonyl]amino]- | and HDAC class II (IIa: HDAC4, 5, |
| 3-pyridinyl]-2-oxoethyl]ethanethioc acid ester) | 7, and 9; IIb: 6 and 10) |
| TCS HDAC6 20b | HDAC6 |
| (2-Methylpropanethioic acid-S-[(6S)-6-[(1,1- | |
| dimethylethoxy)carbonyl]amino]-7-oxo-7-(tricyclo[3.3.1.1^{3,7}]dec-1- | |
| ylamino)heptyl] ester) | |
| NSC 3852 | HDAC |
| (5-Nitroso-8-quinolinol) | |
| NSC69603 | HDAC |
| NSC86371 | HDAC |
| NSC305819 | HDAC |
| CI 994 | HDAC class I |
| (N-acetyldinaline; Acetyldinaline; 4-(Acetylamino)-N-(2- | |
| aminophenyl)benzamide) | |
| LAQ824 | HDAC class I |
| LBH589 | pan-HDAC |
| (panobinostat; Farydak) | |
| MS275 | HDAC1-3 |
| (SNDX-275; entinostat) | |
| MGCD0103 | HDAC1-8 and 11 |
| (mocetinostat) | |
| UF 010 | HDAC1-3 |
| (4-Bromo-N′-butylbenzohydrazide) | |
| Cpd60 | HDAC1-3 |
| Romidepsin | HDAC1 and HDAC2 |
| MS-27-275 | HDAC |
| NaBu | HDAC |
| (n-butyrate) | |
| trapoxin | HDAC |
| Apicidin | HDAC |
| (Cyclo[(2S)-2-Amino-8-oxodecanoyl-1-methoxy-L-tryptophyl-L- | |
| isoleucyl-(2R)-2-piperidinecarbonyl]) | |
| depudesin | HDAC |
| EX 527 | SIRT1 |
| (6-Chloro-2,3,4,9-tetrahydro-1H-carbazole-1-carboxamide) | |
| AGK 2 | SIRT2 |
| (2-Cyano-3-[[5-(2,5-dichlorophenyl)-2-furanyl]-N-5-quinolinyl-2- | |
| propenamide) | |
| AK 7 | SIRT2 |
| (N-(3-Bromophenyl)-3-[(hexahydro-1H-azepin-1- | |
| yl)sulfonyl]benzamide) | |
| SirReal2 | SIRT2 |
| (2-[(4,6-Dimethyl-2-pyrimidinyl)thio]-N-[5-(1- | |
| naphthalenylmethyl)-2-thiazolyl]acetamide) | |
| Salermide | SIRT1 and SIRT2 |
| (N-[3-[[(2-Hydroxy-1-naphthalenyl) | |
| methylene]amino]phenyl]-α-methylbenzeneacetamide) | |
| Splitomicin | Sir2p (yeast form of SIRT1) |
| (1,2-Dihydro-3H-naphtho[2,1-b]pyran-3-one) | |

(b) Inhibitory Nucleic Acids Targeting Fir, Mxi1, and Fbxw7

Aspects of the present disclosure provide methods for reprogramming adult mammalian cochlea to a less differentiated, progenitor state using of one or more inhibitory nucleic acids that target Fir, Mxi1, Fbxw7, or a combination thereof.

The sequences of human Fir, human Mxi1, and human Fbxw7 are known in the art. For example, the sequence of human Fir is available in the NCBI database as NM_078480.3 (SEQ ID NO: 1). For example, the sequence of human Mxi1 is available in the NCBI database as NM_005962.5 (SEQ ID NO: 2). For example, the sequence of human Fbxw7 is available in the NCBI database as NM_033632.3 (SEQ ID NO: 3). See Table 2 below.

TABLE 2
Exemplary sequences of human Fir, human
Mxi1 and human Fbxw7.

| ID | Sequence |
|---|---|
| Human | ATGGCGACGGCGACCATAGCTCTCCAGGTCAATGG |
| Fir | CCAGCAAGGAGGGGGGTCCGAGCCGGCGGCGGCGG |
| (NM_ | CGGCAGTGGTGGCAGCGGGAGACAAATGGAAACCT |
| 078480.3) | CCACAGGGCACAGACTCCATCAAGATGGAGAACGG |
| | GCAGAGCACAGCCGCCAAGCTGGGGCTGCCTCCCC |
| | TGACGCCCGAGCAGCAGGAGGCCCTTCAGAAGGCC |
| | AAGAAGTACGCCATGGAGCAGAGCATCAAGAGTGT |
| | GCTGGTGAAGCAGACCATCGCGCACCAGCAGCAGC |
| | AGCTCACCAACCTGCAGATGGCAGCAGTGACAATG |
| | GGCTTTGGAGATCCTCTCTCACCTTTGCAATCGAT |
| | GGCGGCTCAGCGGCAGCGGGCGCTGGCCATCATGT |
| | GCCGCGTCTACGTGGGCTCTATCTACTATGAGCTG |
| | GGGGAGGACACCATCCGCCAGGCCTTTGCCCCCTT |
| | TGGCCCCATCAAGAGCATCGACATGTCCTGGGACT |
| | CCGTCACCATGAAGCACAAGGGCTTTGCCTTCGTG |
| | GAGTATGAGGTCCCCGAAGCTGCACAGCTGGCCTT |
| | GGAGCAGATGAACTCGGTGATGCTGGGGGGCAGGA |
| | ACATCAAGGTGGGCAGACCCAGCAACATAGGGCAG |
| | GCCCAGCCCATCATAGACCAGTTGGCTGAGGAGGC |
| | ACGGGCCTTCAACCGCATCTACGTGGCCTCTGTGC |
| | ACCAGGACCTCTCAGACGATGACATCAAGAGCGTG |
| | TTTGAGGCCTTTGGCAAGATCAAGTCCTGCACACT |
| | GGCCCGGGACCCCACAACTGGCAAGCACAAGGGCT |
| | ACGGCTTCATTGAGTACGAGAAGGCCCAGTCGTCC |
| | CAAGATGCTGTGTCTTCCATGAACCTCTTTGACCT |
| | GGGTGGCCAGTACTTGCGGGTGGGCAAGGCTGTCA |
| | CACCGCCCATGCCCCTACTCACACCAGCCACGCCT |
| | GGAGGCCTCCCACCTGCCGCTGCTGTGGCAGCTGC |
| | TGCAGCCACTGCCAAGATCACAGCTCAGGAAGCAG |
| | TGGCCGGAGCAGCGGTGCTGGGTACCCTGGGCACA |
| | CCTGGACTGGTGTCCCCAGCACTGACCCTGGCCCA |
| | GCCCCTGGGCACTTTGCCCCAGGCTGTCATGGCTG |
| | CCCAGGCACCTGGAGTCATCACAGGTGTGACCCCA |
| | GCCCGTCCTCCTATCCCGGTCACCATCCCCTCGGT |
| | GGGAGTGGTGAACCCCATCCTGGCCAGCCCTCCAA |
| | CGCTGGGTCTCCTGGAGCCCAAGAAGGAGAAGGAA |
| | GAAGAGGAGCTGTTTCCCGAGTCAGAGCGGCCAGA |
| | GATGCTGAGCGAGCAGGAGCACATGAGCATCTCGG |
| | GCAGTAGCGCCCGACACATGGTGATGCAGAAGCTG |
| | CTCCGCAAGCAGGAGTCTACAGTGATGGTTCTGCG |
| | CAACATGGTGGACCCCAAGGACATCGATGATGACC |
| | TGGAAGGGGAGGTGACAGAGGAGTGTGGCAAGTTC |
| | GGGGCCGTGAACCGCGTCATCATCTACCAAGAGAA |
| | ACAAGGCGAGGAGGAGGATGCAGAAATCATTGTCA |
| | AGATCTTTGTGGAGTTTTCCATAGCCTCTGAGACT |
| | CATAAGGCCATCCAGGCCCTCAATGGCCGCTGGTT |
| | TGCTGGCCGCAAGGTGGTGGCTGAAGTGTACGACC |
| | AGGAGCGTTTTGATAACAGTGACCTCTCTGCGTGA |
| | (SEQ ID NO: 1) |
| Human | ATGGAGCGGGTGAAGATGATCAACGTGCAGCGTCT |
| Mxi1 | GCTGGAGGCTGCCGAGTTTTTGGAGCGCCGGGAGC |
| (NM_ | GAGAGTGTGAACATGGCTACGCCTCTTCATTCCCG |
| 005962.5) | TCCATGCCGAGCCCCCGACTGCAGCATTCAAAGCC |
| | CCCACGGAGGTTGAGCCGGGCACAGAAACACAGCA |
| | GCGGGAGCAGCAACACCAGCACTGCCAACAGATCT |
| | ACACACAATGAGCTGGAAAAGAATCGACGAGCTCA |
| | TCTGCGCCTTTGTTTAGAACGCTTAAAAGTTCTGA |
| | TTCCACTAGGACCAGACTGCACCCGGCACACAACA |
| | CTTGGTTTGCTCAACAAAGCCAAAGCACACATCAA |
| | GAAACTTGAAGAAGCTGAAAGAAAAAGCCAGCACC |
| | AGCTCGAGAATTTGGAACGAGAACAGAGATTTTTA |
| | AAGTGGCGACTGGAACAGCTGCAGGGTCCTCAGGA |
| | GATGGAACGAATACGAATGGACAGCATTGGATCAA |
| | CTATTTCTTCAGATCGTTCTGATTCAGAGCGAGAG |
| | GAGATTGAAGTGGATGTTGAAAGCACAGAGTTCTC |
| | CCATGGAGAAGTGGACAATATAAGTACCACCAGCA |
| | TCAGTGACATTGATGACCACAGCAGCCTGCCGAGT |
| | ATTGGGAGTGACGAGGGTTACTCCAGTGCCAGTGT |
| | CAAACTTTCATTCACTTCATAG |
| | (SEQ ID NO: 2) |
| Human | ATGAATCAGGAACTGCTCTCTGTGGGCAGCAAAAG |
| Fbxw7 | ACGACGAACTGGAGGCTCTCTGAGAGGTAACCCTT |
| (NM_ | CCTCAAGCCAGGTAGATGAAGAACAGATGAATCGT |
| 033632.3) | GTGGTAGAGGAGGAACAGCAACAGCAACTCAGACA |
| | ACAAGAGGAGGAGCACACTGCAAGGAATGGTGAAG |
| | TTGTTGGAGTAGAACCTAGACCTGGAGGCCAAAAT |
| | GATTCCCAGCAAGGACAGTTGGAAGAAAACAATAA |
| | TAGATTTATTTCGGTAGATGAGGACTCCTCAGGAA |
| | ACCAAGAAGAACAAGAGGAAGATGAAGAACATGCT |
| | GGTGAACAAGATGAGGAGGATGAGGAGGAGGAGGA |
| | GATGGACCAGGAGAGTGACGATTTTGATCAGTCTG |
| | ATGATAGTAGCAGAGAAGATGAACATACACATACT |
| | AACAGTGTCACGAACTCCAGTAGTATTGTGGACCT |
| | GCCCGTTCACCAACTCTCCTCCCCATTCTATACAA |
| | AAACAACAAAAATGAAAAGAAAGTTGGACCATGGT |
| | TCTGAGGTCCGCTCTTTTTCTTTGGGAAAGAAACC |
| | ATGCAAAGTCTCAGAATATACAAGTACCACTGGGC |
| | TTGTACCATGTTCAGCAACACCAACAACTTTTGGG |
| | GACCTCAGAGCAGCCAATGGCCAAGGGCAACAACG |
| | ACGCCGAATTACATCTGTCCAGCCACCTACAGGCC |
| | TCCAGGAATGGCTAAAAATGTTTCAGAGCTGGAGT |
| | GGACCAGAGAAATTGCTTGCTTTAGATGAACTCAT |
| | TGATAGTTGTGAACCAACACAAGTAAAACATATGA |
| | TGCAAGTGATAGAACCCCAGTTTCAACGAGACTTC |
| | ATTTCATTGCTCCCTAAAGAGTTGGCACTCTATGT |
| | GCTTTCATTCCTGGAACCCAAAGACCTGCTACAAG |
| | CAGCTCAGACATGTCGCTACTGGAGAATTTTGGCT |
| | GAAGACAACCTTCTCTGGAGAGAGAAATGCAAAGA |
| | AGAGGGGATTGATGAACCATTGCACATCAAGAGAA |
| | GAAAAGTAATAAAACCAGGTTTCATACACAGTCCA |
| | TGGAAAAGTGCATACATCAGACAGCACAGAATTGA |
| | TACTAACTGGAGGCGAGGAGAACTCAAATCTCCTA |
| | AGGTGCTGAAAGGACATGATGATCATGTGATCACA |
| | TGCTTACAGTTTTGTGGTAACCGAATAGTTAGTGG |
| | TTCTGATGACAACACTTTAAAAGTTTGGTCAGCAG |
| | TCACAGGCAAATGTCTGAGAACATTAGTGGGACAT |
| | ACAGGTGGAGTATGGTCATCACAAATGAGAGACAA |
| | CATCATCATTAGTGGATCTACAGATCGGACACTCA |
| | AAGTGTGGAATGCAGAGACTGGAGAATGTATACAC |
| | ACCTTATATGGGCATACTTCCACTGTGCGTTGTAT |
| | GCATCTTCATGAAAAAAGAGTTGTTAGCGGTTCTC |
| | GAGATGCCACTCTTAGGGTTTGGGATATTGAGACA |
| | GGCCAGTGTTTACATGTTTTGATGGGTCATGTTGC |
| | AGCAGTCCGCTGTGTTCAATATGATGGCAGGAGGG |
| | TTGTTAGTGGAGCATATGATTTTATGGTAAAGGTG |
| | TGGGATCCAGAGACTGAAACCTGTCTACACACGTT |
| | GCAGGGGCATACTAATAGAGTCTATTCATTACAGT |
| | TTGATGGTATCCATGTGGTGAGTGGATCTCTTGAT |
| | ACATCAATCCGTGTTTGGGATGTGGAGACAGGGAA |
| | TTGCATTCACACGTTAACAGGGCACCAGTCGTTAA |
| | CAAGTGGAATGGAACTCAAAGACAATATTCTTGTC |
| | TCTGGGAATGCAGATTCTACAGTTAAAATCTGGGA |
| | TATCAAAACAGGACAGTGTTTACAAACATTGCAAG |
| | GTCCCAACAAGCATCAGAGTGCTGTGACCTGTTTA |
| | CAGTTCAACAAGAACTTTGTAATTACCAGCTCAGA |
| | TGATGGAACTGTAAAACTATGGGACTTGAAAACGG |
| | GTGAATTTATTCGAAACCTAGTCACATTGGAGAGT |
| | GGGGGGAGTGGGGGAGTTGTGTGGCGGATCAGAGC |
| | CTCAAACACAAAGCTGGTGTGTGCAGTTGGGAGTC |
| | GGAATGGGACTGAAGAAACCAAGCTGCTGGTGCTG |
| | GACTTTGATGTGGACATGAAGTGA |
| | (SEQ ID NO: 3) |

As used herein, an “inhibitory nucleic acid” refers to a nucleic acid, or a mimetic thereof, that when administered to a mammalian cell results in a decrease in the expression of a target gene. Typically, an inhibitory nucleic acid comprises at least a portion of a target nucleic acid molecule that hybridizes to at least a portion of the target nucleic acid and modulates its function. In some embodiments, expression of a target gene is reduced by at least 10%, at least 25%, at least 50%, at least 75%, at least 90%, or at least 95% or more.

Examples of inhibitory nucleic acids include, but are not limited to, short interfering RNA (siRNA), short hairpin RNA (shRNA), and antisense oligonucleotides. Inhibitory nucleic acids can include DNA, RNA, modified nucleic acids, and combinations thereof (e.g., DNA/RNA hybrids). Methods for making and delivering inhibitory nucleic acids that target a specific sequence are known in the art, see, e.g., Ramachandran and Ignacimuthu, Appl Biochem Biotechnol. 2013; 169(6):1774-89; Li et al., J Control Release 2013; 172(2):589-600; Lochmatter and Mullis, Horm Res Paediatr. 2011; 75(1):63-9; Higuchi et al., BioDrugs. 2010; 24(3):195-205; and Ming et al., Expert Opin Drug Deliv. 2011; 8(4):435-49.

Methods described herein encompass use of a single inhibitory nucleic acid or multiple inhibitory nucleic acids. For example, when targeting Fir and Mxi1, methods and compositions described herein can include use of a single inhibitory nucleic acid that targets Fir and Mxi1 or use of an inhibitory nucleic acid that targets Fir and an inhibitory nucleic acid that targets Mxi1.

In some embodiments, the inhibitory nucleic acid targets one of Fir, Mxi1, or Fbxw7 (e.g., the inhibitory nucleic acid targets Fir, the inhibitory nucleic acid targets Mxi1, or the inhibitory nucleic acid targets Fbxw7). In some embodiments, the inhibitory nucleic acid targets two of Fir, Mxi1, and Fbxw7 (e.g., the inhibitory nucleic acid targets Fir and Mxi1, the inhibitory nucleic acid targets Fir and Fbxw7, or the inhibitory nucleic acid targets Mxi1 and Fbxw7). In some embodiments, the inhibitory nucleic acid targets each one of Fir, Mxi1, and Fbxw7.

(c) Wnt Agonists and cAMP Agonists

Methods described herein, in some embodiments, involve use of a Wnt agonist and/or a cAMP agonist for reprogramming adult mammalian cochlea to a less differentiated, progenitor state.

As used herein, the term “Wnt agonist” refers to any agent that activates the Wnt/0-catenin pathway, or inhibits the activity and/or expression of inhibitors of Wnt/0-catenin signaling, for example antagonists or inhibitors of GSK-3β activity.

Wnt agonists include Wnt proteins or other compounds that bind directly to the Frizzled and lipoprotein receptor-related protein 5/6 (LRP5/6) co-receptor proteins (e.g., a Frizzled receptor activator, a LRP5/6 activator), in manner that promotes an increase in the concentration of β-catenin in the nucleus of a mammalian cell. Alternatively, a Wnt agonist may function by inhibiting one or more secreted Frizzled-related proteins (sFRPs) (e.g., an sFRP inhibitor) or Wnt inhibitory protein (WIF) (e.g., a WIF inhibitor), which bind and sequester Wnt proteins from interacting with the endogenous Wnt co-receptors. Examples of Wnt agonists also include, but are not limited to, a glycogen synthase kinase-30 (GSK-30) inhibitor, a Wnt activator, a Disheveled (Dvl) activator, an Axin inhibitor, a Dickkopf (Dkk) inhibitor, and a Groucho inhibitor. GSK-30 is a kinase that forms a complex with Axin, APC (Adenomatous polyposis coli), and β-catenin to prepare β-catenin for downstream degradation by the proteasome. Disheveled (Dvl) is an intracellular protein that relays signals from activated Notch receptors to downstream effectors. Dvl is recruited by the receptor Frizzled and prevents the constitutive descruction of β-catenin. Dkk is a secreted protein that acts to isolate the LRP5/6 co-receptor proteins, thus inhibiting Wnt signaling. Groucho is a protein that forms a complex with TLE in the nucleus to repress gene expression. Once β-catenin enters the nucleus, it disrupts the Groucho/TLE complex to activate gene expression. A Wnt agonist can be a small molecule compound that activates Wnt signaling, e.g., see WO2018172997, the relevant disclosures of which are incorporated by reference for the purpose and subject matter referenced herein. Examples of Wnt agonists include, but are not limited to, compounds listed in Table 3.

TABLE 3
Exemplary Wnt agonists.

| Compound | Target |
|---|---|
| Lithium chloride (LiCl) | GSK-3β |
| CHIR-99021 | GSK-3β |
| CHIR-98023 | GSK-3β |
| CHIR-99030 | GSK-3β |
| Hymenialdisine | GSK-3β |
| debromohymeialdisine | GSK-3β |
| dibromocantherelline | GSK-3β |
| Meridianine A | GSK-3β |
| alsterpaullone | GSK-3β |
| cazapaullone | GSK-3β |
| Aloisine A | GSK-3β |
| NSC 693868 | GSK-3β |
| (1H-Pyrazolo[3,4-b]quinoxalin-3-amine) | |
| Indirubin-3′-oxime | GSK-3β |
| (Indirubin-3′-monoxime; 3-[1,3-Dihydro-3- | |
| (hydroxyimino)-2H-indol-2-ylidene]- | |
| 1,3-dihydro-2H-indol-2-one) | |
| A 1070722 | GSK-3β |
| (1-(7-Methoxyquinolin-4-yl)-3-[6- | |
| (trifluoromethyl)pyridin-2-yl]urea) | |
| L803 | GSK-3β |
| L803-mts | GSK-3β |
| TDZD8 | GSK-3β |
| NP00111 | GSK-3β |
| HMK-32 | GSK-3β |
| Manzamine A | GSK-3β |
| Palinurin | GSK-3β |
| Tricantin | GSK-3β |
| IM-12 | GSK-3β |
| (3-(4-Fluorophenylethylamino)-1- | |
| methyl-4-(2-methyl-1H-indol-3- | |
| yl)-1H-pyrrole-2,5-dione) | |
| NP031112 | GSK-3β |
| NP00111 | GSK-3β |
| NP031115 | GSK-3β |
| VP 2.51 | GSK-3β |
| VP2.54 | GSK-3β |
| VP 3.16 | GSK-3β |
| VP 3.35 | GSK-3β |
| HLY78 | Axin |
| (4-Ethyl-5,6-Dihydro-5-methyl- | |
| [1,3]dioxolo[4,5- | |
| j]phenanthridine, 4-Ethyl- | |
| 5-methyl-5,6-dihydro- | |
| [1,3]dioxolo[4,5- | |
| j]phenanthridine) | |
| WAY-262611 | Dickkopf-1 (DKK1) |
| ((1-(4-(Naphthalen-2-yl)pyrimidin-2- | |
| yl)piperidin-4-yl)methanamine)) | |
| BHQ880 | DKK1 |
| NCI8642 | DKK1 |
| gallocyanine dyes | DKK1 |
| Compounds 3-8 | secreted frizzled-related |
| (Moore et al., J. Med. Chem., 2009; 52: 105) | protein 1 (sFRP-1) |
| WAY-316606 | sFRP-1 |

As used herein, a “cAMP agonist” refers to an agent that increases intracellular levels of cAMP as compared to the background physiological intracellular level when the agent is absent. Examples of cAMP agonists include, but are not limited to, forskolin, rolipram, NKH477, PACAP1-27, PACAP1-38, and isoproterenol.

(d) Atoh1 Activators

After reprogramming adult wild-type cochlea to a less differentiated state, Atoh1 activity can be increased to regenerate HCs in the adult mammalian cochlea. Accordingly, methods described herein, in some embodiments, involve use of agents that increase Atoh1 activity to regenerate cochlear hair cells in adult wild-type cochlea that have been reprogrammed to a less differentiated state (e.g., reprogramed to re-express inner ear progenitor genes).

As used herein, the term “Atoh1” refers to a protein belonging to the basic helix-loop-helix (BHLH) family of transcription factors that is involved in the formation of hair cells in an inner ear of a mammal.

Any method of increasing Atoh1 activity can be used in methods described herein. Methods of increasing Atoh1 activity (including use of Atoh1 agonists) are known in the art (see, e.g., U.S. Pat. No. 8,188,131; U.S. Patent Publication No. 20110305674; U.S. Patent Publication No. 20090232780; Kwan et al. (2009) INT'L SYMPOSIUM ON OLFACTION AND TASTE: ANN. N.Y. ACAD. SCI. 1170:28-33; Daudet et al. (2009) Dev. Bio. 326:86-100; Takebayashi et al. (2007) Dev. Bio. 307:165-178; and Ahmed et al. (2012) Dev. Cell 22(2):377-390).

Non-limiting examples of methods for increasing Atoh1 activity that can be used in methods described herein are provided in U.S. Patent Publication No. US 2020/0338160 A1, which is incorporated by reference herein in its entirety.

An agent for increasing Atoh1 activity can be a nucleic acid encoding Atoh1, an Atoh1 protein, or an Atoh1 agonist. Exemplary Atoh1 polypeptides include, for example, NP_005163.1, as referenced in the NCBI protein database. Exemplary Atoh1 nucleic acid sequences that may be expressed in target cells include, for example, NM_005172.1, as referenced in the NCBI gene database.

Other exemplary Atoh1 activators described in U.S. Pat. No. 8,188,131 include 4-(4-chlorophenyl)-1-(5H-pyrimido[5,4-b]indol-4-yl)-1H-pyrazol-3-amine; 6-chloro-1-(2-chlorobenzyloxy)-2-phenyl-1H-benzo[d]imidazole; 6-chloro-1-(2-chlorobenzyloxy)-2-(4-methoxyphenyl)-1H-benzo[d]imidazole; 6-chloro-2-(4-methoxyphenyl)-1-(4-methylbenzyloxy)-1H-benzo[d]imidazole; 6-chloro-1-(3,5-dimethylbenzyloxy)-2-(4-methoxyphenyl)-1H-benzo[d]imidazole; 6-chloro-1-(4-methoxybenzyloxy)-2-(4-methoxyphenyl)-1H-benzo[d]imidazole; 1-(4-methylbenzyloxy)-6-nitro-2-phenyl-1H-benzo[d]imidazole; 4-(1H-benzo[d]imidazol-2-yl)phenol; 2,5-dichloro-N-((1-methyl-H-benzo[d]imidazol-2-yl)methyl)aniline; 4-(2-(1-methyl-1H-benzo[d]imidazol-2-yl)ethyl)aniline; 2-((2-methoxyphenoxy)methyl)-1H-benzo[d]imidazole; 2-((4-fluorophenoxy)methyl)-1-methyl-1H-benzo[d]imidazole; 2-(phenylthiomethyl)-1H-benzo[d]imidazole; 3-(6-methyl-1H-benzo[d]imidazole-2-yl)-2H-chromen-2-imine; N-(2-(1H-benzo[d]imidazole-2-yl)phenyl)isobutyramide; 2-(o-tolyloxymethyl)-1H-benzo[d]imidazole; 2-(4-methoxyphenyl)-1-phenethyl-1H-benzo[d]imidazole; N-(6-bromobenzo[d]thiazole-2-yl)thiophene-2-carboxamide; N-(benzo[d]thiazole-2-yl)-1-methyl-1H-pyrazole-5-carboxamide; 2-(4-fluorobenzylthio)benzo[d]thiazole; 5-chloro-N-methylbenzo[d]thiazole-2-amine; N-(6-acetamidobenzo[d]thiazol-2-yl)furan-2-carboxamide; N-(6-fluorobenzo[d]thiazole-2-yl)-3-methoxybenzamide; 2-(benzo[d]oxazol-2-ylthio)-N-(2-chlorophenyl)acetamide; 5-chloro-2-phenylbenzo[d]oxazole; 5-methyl-2-m-tolylbenzo[d]oxazole; 2-(4-isobutoxyphenyl)-3-(naphthalen-2-yl)-2,3-dihydroquinazolin-4(1H)-one; N-(2-(2-(4-fluorophenyl)-2-oxoethylthio)-4-oxoquinazolin-3(4H)-yl)benzamide; 2-(4-chlorophenyl)-4-(4-methoxyphenyl)-1,4-dihydrobenzo[4,5]imidazo[1,2-a]pyrimidine; 2-(3-pyridyl)-4-(4-bromophenyl)-1,4-dihydrobenzo[4,5]imidazo[1,2-a]pyrimidine; N-sec-butyl-1,7,7-trimethyl-9-oxo-8,9-dihydro-7H-furo[3,2-f]chromene-2-carboxamide; N-(3-carbamoyl-5,6-dihydro-4H-cyclopenta[b]thiophen-2-yl)benzofuran-2-carboxamide; 3-chloro-N-(5-chloropyridin-2-yl)benzo[b]thiophene-2-carboxamide; 3-chloro-N-((tetrahydrofuran-2-yl)methyl)benzo[b]thiophene-2-carboxamide; N-(3-(5-chloro-3-methylbenzo[b]thiopen-2-yl)-1H-pyrazol-5-yl)acetamide; 2-(naphthalen-2-yl)-1H-indole; 2-(pyridin-2-yl)-1H-indole; N-(2-chlorophenyl)-2-(1H-indole-3-yl)-2-oxoacetamide; 2-m-tolylquinoline; 2-(4-(2-methoxyphenyl) piperazin-1-yl)quinolone; 2-(1H-benzo[d][1,2,3]triazol-1-yl)-N-(2,3-dihydro-1H-inden-2-yl)acetamide; 1-phenethyl-1H-benzo[d][1,2,3]triazole; 7-(4-fluorobenzyloxy)-2H-chromen-2-one; N-(2,4-dichlorophenyl)-8-methoxy-2H-chromene-3-carboxamide; N-(3-chlorophenyl)-8-methyl-3,4-dihydroquinoline-1(2H)-carbothioamide; 7-methoxy-5-methyl-2-phenyl-4H-chromen-4-one; 2-(3,4-dimethylphenyl)quinoxaline; 4-bromo-N-(5-chloropyridin-2-yl)benzamide; 3-amino-6,7,8,9-tetrahydro-5H-cyclohepta[e]thieno[2,3-b]pyridine-2-carboxamide; (Z)-3-methyl-N′-(nicotinoyloxy)benzimidamide; N,N-diethyl-6-methoxythieno[2,3-b]quinoline-2-carboxamide; 6-(4-methoxyphenyl)-1,2,3,4-tetrahydro-1,5-naphthyridine; 5-bromo-N-(2-(phenylthio)ethyl) nicotinamide; N-(6-methylpyridin-2-yl)-2,3-dihydrobenzo[b][1,4]dioxine-6-carboxamide; 2-(4-methylbenzylthio)oxazolo[4,5-b]pyridine; N-(2-methoxyethyl)-5-p-tolylpyrimidin-2-amine; 4-(5-(benzo[b]thiophen-2-yl)pyrimidin-2-yl)morpholino; 4-(5-(4-fluorophenyl)pyrimidin-2-yl)morpholino; N-(4-bromo-3-methylphenyl)quinazoline-4-amine; N-(4-methoxyphenyl)quinazolin-4-amine; N-(3-methoxyphenyl)-9H-purin-6-amine; N,N-diethyl-1-m-tolyl-1H-pyrazolo[3,4-d]pyrimidin-4-amine; (5-(4-bromophenyl)furan-2-yl)(morpholino)methanone; (Z)-4-bromo-N′-(furan-2-carbonyloxy)benzimidamide; N-(4-iodophenyl)furan-2-carboxamide; 5-(5-(2,4-difluorophenyl)furan-2-yl)-1-(methylsulfonyl)-1H-pyrazole; 1-(3-amino-5-(4-tert-butylphenyl)thiophen-2-yl)ethanone; N-(3-cayano-4,5,6,7-tetrahydrobenzo[b]thiophen-2-yl)-2-fluorobenzamide; N-(5-chloropyridin-2-yl)thiophene-2-carboxamide; N-(2-(4-fluorophenoxy)ethyl)thiophene-2-carboxamide; 2,5-dimethyl-N-phenyl-1-(thiophen-2-ylmethyl)-1H-pyrrole-3-carboxamide; N-(3-cyanothiophen-2-yl)-4-isopropoxybenzamide; 2-(4-methoxyphenoxy)-N-(thiazol-2-yl)acetamide; 4-(4-methoxyphenyl)-N-(3-methylpyridin-2-yl)thiazol-2-amine; 4-(biphenyl-4-yl)thiazol-2-amine; 4-(4-(4-methoxyphenyl)thiazol-2-yl)-3-methylisoxazol-5-amine; N-(2-methoxyphenyl)-4-phenylthiazol-2-amine; 1-(4-amino-2-(m-tolylamino)thiazol-5-yl)-2-methylpropan-1-one; 4-(4-chlorophenyl)-1-(5H-pyrimido[5,4-b]indol-4-yl)-1H-pyrazol-3-amine; 2-(4-chlorophenyl)-6-ethyl-5-methylpyrazolo[1,5-a]pyrimidin-7(4H)-one; 5-methoxy-2-(5-phenyl-1H-pyrazol-3-yl)phenol; (3-(4-bromophenyl)-1-phenyl-1H-pyrazol-4-yl)methanol; N-(2,5-dichlorophenyl)-1-ethyl-1H-pyrazole-3-carboxamide; 4-chloro-1-methyl-N-(2-oxo-2-phenylethyl)-1H-pyrazole-3-carboxamide; N-(3-(5-tert-butyl-2-methylfuran-3-yl)-1H-pyrazole-5-yl)benzamide; N-(5-methylisoxazol-3-yl)benzo[d][1,3]dioxole-5-carboxamide; (5-(4-bromophenyl)isoxazole-3-yl)(morpholino)methanone; N-(4-bromophenyl)-5-isopropylisoxazole-3-carboxamide; 5-((4-chloro-2-methylphenoxy)methyl)-3-(pyridin-4-yl)-1,2,4-oxadiazole; 5-(2-methoxyphenyl)-3-p-tolyl-1,2,4-oxadiazole; 5-(phenoxymethyl)-3-(pyridin-3-yl)-1,2,4-oxadiazole; 5-(2-chloro-4-methylphenyl)-3-(pyridin-3-yl)-1,2,4-oxadiazole; 3-(2-chlorophenyl)-5-p-tolyl-1,2,4-oxadiazole; 5-(piperidin-1-ylmethyl)-3-p-toyl-1,2,4-oxadiazole; 5-(4-bromophenyl)-3-(pyridin-3-yl)-1,2,4-oxadiazole; 5-(2-bromophenyl)-3-(4-bromophenyl)-1,2,4-oxadiazole; 5-(2-bromo-5-methoxyphenyl)-3-(thiophenyl-2-yl)-1,2,4-oxadiazole; 3-(2-fluorophenyl)-N-(3-(piperidin-1-yl)propyl)-1,2,4-oxadiazol-5-amine; 2-(2-chlorobenzoyl)-N-(4-fluorophenyl)hydrazinecarbothioamide; 2-(methylamino)-N-phenethylbenzamide; 4-tert-butyl-N-((tetrahydrofuran-2-yl)methyl)benzamide; 2-phenyl-5-o-tolyl-1,3,4-oxadiazole; 4-(3-(4-chlorophenyl)-4,5-dihydro-1H-1,2,4-triazole-5-yl)-N,N-dimethylaniline; 7-methoxy-2-(4-methoxyphenyl)-1,10b-dihydrospiro[benzo[e]pyrazolo[1,5-c][1,3]oxazine-5,1′-cyclohexane]; 6-oxo-2-(4-(3-(trifluoromethyl)phenoxy)phenyl)-1,4,5,6-tetrahydropyridine-3-carbonitrile; 6-(4-methoxyphenyl)imidazo[2,1-b]thiazole; 2-(2-bromophenoxy)-N-(4H-1,2,4-triazol-3-yl)acetamide; 1-(indolin-1-yl)-2-phenoxyethanone; 2-(4-chlorophenyl)-6,7,8,9-tetrahydrobenzo[e]imidazo[1,2-b][1,2,4]triazine; and pharmaceutically acceptable salts thereof.

(e) Pharmaceutical Compositions

Any of the compounds for regenerating cochlear hair cells described herein can be mixed with a pharmaceutically acceptable carrier or an excipient to form a pharmaceutical composition for use in methods of producing a population of cochlear progenitor cells and methods of treating hearing loss.

Pharmaceutical compositions comprising one or more compounds as described herein can be formulated according to the intended method of administration. For example, a pharmaceutical composition can be formulated for local or systemic administration, e.g., administration by drops (e.g., otic drops) or injection into the ear, insufflation (such as into the ear), intravenous, topical, or oral administration. One or more compounds as described herein can be formulated as pharmaceutical compositions for direct administration to a subject.

The nature of the pharmaceutical compositions for administration is dependent on the mode of administration and can readily be determined by one of ordinary skill in the art. In some embodiments, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral.

A pharmaceutical composition can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, e.g., Remington: The Science and Practice of Pharmacy, 22^{nd} Ed., Allen, ed., Mack Publishing Co., Easton, Pa., 2012.

One or more of the compounds can be administered, e.g., as a pharmaceutical composition, directly and/or locally by injection or through surgical placement, e.g., to the inner ear. The amount of the pharmaceutical composition may be described as the effective amount or as a therapeutically effective amount. Where application over a period of time is advisable or desirable, the compositions of the invention can be placed in sustained released formulations or implantable devices (e.g., a pump).

Alternatively or in addition, the pharmaceutical compositions can be formulated for systemic parenteral administration by injection, for example, by bolus injection or continuous infusion. Such formulations can be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, for example, sterile pyrogen-free water, before use.

In addition to the formulations described previously, the compositions can also be formulated as a depot preparation. Such long acting formulations can be administered by implantation (e.g., subcutaneously). Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

Pharmaceutical compositions formulated for systemic oral administration can take the form of tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets can be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

In some embodiments, the pharmaceutical compositions described herein can include one or more of the compounds formulated according to any of the methods described herein.

### II. Methods of Reprogramming Mammalian Cochlea for Hair Cell Regeneration

Aspects of the present disclosure provide methods for reprogramming adult mammalian cochlea that involve contacting the mammalian cochlea with a HDAC inhibitor and one or more inhibitory nucleic acids that target Fir, Mxi1, Fbxw7, or a combination thereof, under conditions and for a time sufficient to produce a population of cochlear progenitor cells. In some embodiments, methods described herein further comprise contacting the mammalian cochlea with a Wnt agonist (e.g., LiCl) and/or a cAMP agonist (e.g., forskolin).

As used herein, the term “reprogramming” refers to a process that alters or reverses the differentiation state of a cochlear cell. For example, an adult mammalian cochlea including differentiated cochlear cells can be reprogrammed such that differentiated cochlear cells are converted to cochlear progenitor cells. Reprogramming encompasses complete or partial reversion of the differentiation state of a cochlea cell. In some embodiments, reprogramming renders the mammalian cochlea or the mammalian cochlear cell more susceptible to regenerating hair cells when contacted with an Atoh1 activator.

As used herein, the term “progenitor cell” refers to an immature or undifferentiated cell that has the potential to mature (differentiate) into a specific cell type, for example, a hair cell. As used herein, “cochlear progenitor cell” refers to a progenitor cell with the differentiation potential to form a cochlear hair cell. Progenitor cells have a cellular phenotype that is more primitive than a cell which it can give rise to by differentiation. For example, cochlear progenitor cells can express progenitor markers such as Six1, Eya1, Gata3, Sox2, Notch1, Hes5, or a combination.

To perform the reprogramming methods described herein, adult mammalian cochlea are contacted with a combination of agents such as an HDAC inhibitor and one or more inhibitory nucleic acids. As used herein, the term “contacting” refers to an exposure of a mammalian cochlea with a combination of agents for hair cell regeneration (e.g., a HDAC inhibitor and one or more inhibitory nucleic acids targeting Fir, Mxi1, Fbxw7, or a combination thereof, and optionally a Wnt agonist and/or a cAMP agonist) under conditions and for a time sufficient to produce a population of cochlear progenitor cells in the mammalian cochlea. The mammalian cochlea can be contacted with the combination of agents for hair cell regeneration in vitro (e.g., in a culture) or in vivo (e.g., in a subject).

Methods described herein encompass contacting a mammalian cochlea with a combination of agents described herein for a time suitable for reprogramming cochlear cells to a less differentiated, progenitor state. In some embodiments, methods described herein comprise contacting a mammalian cochlea with a combination of agents described herein for 1-15 days, e.g., 2-15 days, 3-15 days, 4-15 days, 5-15 days, 6-15 days, 7-15 days, 8-15 days, 9-15 days, 10-15 days, 11-15 days, 12-15 days, 13-15 days, 14-15 days, 1-14 days, 1-13 days, 1-12 days, 1-11 days, 1-10 days, 1-9 days, 1-8 days, 1-7 days, 1-6 days, 1-5 days, 1-4 days, 1-3 days, or 1-2 days.

The presence of cochlear progenitor cells can be determined using any method known in the art, for example, by detecting expression of one or more progenitor genes such as Six1, Eya1, Gata3, Sox2, Notch1, Hes5, or a combination thereof in the mammalian cochlea.

### III. Methods of Treatment

Provided herein are methods of treating hearing loss in a mammalian subject (e.g., a human or a non-human veterinary subject) using a HDAC inhibitor and one or more inhibitory nucleic acids that target Fir, Mxi1, Fbxw7, or a combination thereof, and optionally a Wnt agonist and/or a cAMP agonist. In some embodiments the subject is post-neonatal (e.g., a child, an adolescent or an adult, e.g., above the age of 1, 2, 3, 4, 5, 6, 7, 8, 9, 10, 11, 12 or 13 years) subject. The subject can receive treatment with a combination of agents as described herein.

Methods described herein encompass treating hearing loss and any disorder that arises as a consequence of cochlear hair cell loss such as noise-induced permanent deafness, drug-induced hearing loss, age-related hearing loss, vestibular dysfunction.

In some embodiments, the hearing loss is sensorineural hearing loss, which can result from damage or malfunction of the cochlea, e.g., loss of or damage to the sensory epithelium resulting in loss of hair cells.

In some embodiments, the hearing loss can be for any reason, or as a result of any type of event. For example, because of a genetic or congenital defect; for example, a human subject can have been deaf since birth, or can be deaf or hard-of-hearing as a result of a gradual loss of hearing due to a genetic or congenital defect. In another example, the hearing loss can be a result of a traumatic event, such as a physical trauma to a structure of the ear, or a sudden loud noise, or a prolonged exposure to loud noises. For example, prolonged exposures to concert venues, airport runways, and construction areas can cause inner ear damage and subsequent hearing loss.

In some embodiments, hearing loss can be due to chemical-induced ototoxicity, wherein ototoxins include therapeutic drugs including antineoplastic agents, salicylates, quinines, and aminoglycoside antibiotics, contaminants in foods or medicinals, and environmental or industrial pollutants. In some embodiments, hearing loss can result from aging.

In some embodiments, methods include selecting a subject. Subjects suitable for treatment include those having or at risk of having inner ear hair cell loss or those having or at risk of having sensorineural hearing loss. Any subject experiencing or at risk for developing hearing loss is a candidate for the treatment methods described herein. In some examples, the subject has noise-induced permanent deafness, drug-induced hearing loss, age-related hearing loss, sudden sensorineural hearing loss, hearing loss due to viral infection, tinnitus, vestibular dysfunction, or a combination thereof. A human subject having or at risk for developing a hearing loss can hear less well than the average human being, or less well than a human before experiencing the hearing loss. For example, hearing can be diminished by at least 5, 10, 30, 50% or more.

In some embodiments, the methods include administering to the subject a combination of agents described herein within one, two, three, four, five, six, or seven days, or one, two, three, four, five, or six weeks of exposure to an ototoxic insult, e.g., a physical (noise, trauma) or chemical (ototoxin) insult that results in or could result in a loss of hair cells.

In some embodiments, a subject suitable for the treatment using the agents and methods featured in the invention can include a subject having a vestibular dysfunction, including bilateral and unilateral vestibular dysfunction; the methods include administering a therapeutically effective amount of an agent described herein, e.g., by systemic administration or administration via the endolymphatic sac (ES). Vestibular dysfunction is an inner ear dysfunction characterized by symptoms that include dizziness, imbalance, vertigo, nausea, and fuzzy vision and may be accompanied by hearing problems, fatigue and changes in cognitive functioning. Vestibular dysfunctions that can be treated by the methods described herein can be the result of a genetic or congenital defect; an infection, such as a viral or bacterial infection; or an injury, such as a traumatic or nontraumatic injury, that results in a loss of vestibular hair cells. In some embodiments, balance disorders or Meniere's disease (idiopathic endolymphatic hydrops) may be treated by the methods described herein. Vestibular dysfunction is most commonly tested by measuring individual symptoms of the disorder (e.g., vertigo, nausea, and fuzzy vision).

Alternatively or in addition, the agents and methods featured in the invention can be used prophylactically, such as to prevent, reduce or delay progression of hearing loss, deafness, or other auditory disorders associated with loss of hair cells. For example, a composition containing one or more agents can be administered with (e.g., before, after or concurrently with) an ototoxic therapy, i.e., a therapeutic that has a risk of hair cell toxicity and thus a risk of causing a hearing disorder. Ototoxic drugs include the antibiotics neomycin, kanamycin, amikacin, viomycin, gentamycin, tobramycin, erythromycin, vancomycin, and streptomycin; chemotherapeutics such as cisplatin; nonsteroidal anti-inflammatory drugs (NSAIDs) such as choline magnesium trisalicylate, diclofenac, diflunisal, fenoprofen, flurbiprofen, ibuprofen, indomethacin, ketoprofen, meclofenamate, nabumetone, naproxen, oxaprozin, phenylbutazone, piroxicam, salsalate, sulindac, and tolmetin; diuretics; salicylates such as aspirin; and certain malaria treatments such as quinine and chloroquine. For example, a subject undergoing chemotherapy can be treated using the agents and methods described herein. The chemotherapeutic agent cisplatin, for example, is known to cause hearing loss. Therefore, a composition containing one or more agents can be administered with cisplatin therapy (e.g., before, after or concurrently with) to prevent or lessen the severity of the cisplatin side effect. Such a composition can be administered before, after and/or simultaneously with the second therapeutic agent. The two agents may be administered by the same route or by different routes.

In general, the agents and methods described herein can be used to generate hair cell growth and/or to increase the number of hair cells in the inner ear (e.g., the cochlea and/or the utricle). For example, the number of hair cells in the inner ear (e.g., the cochlea and/or the utricle) can be increased about 2-, 3-, 4-, 6-, 8-, or 10-fold, or more, as compared to the number of hair cells before treatment. This new hair cell growth can effectively restore or establish at least a partial improvement in the subject's ability to hear. For example, administration of an agent can improve hearing loss by about 5, 10, 15, 20, 40, 60, 80, 100% or more.

In some instances, compositions can be administered to a subject, e.g., a subject identified as being in need of treatment, using a systemic route of administration. Systemic routes of administration can include, but are not limited to, parenteral routes of administration, e.g., intravenous injection, intramuscular injection, and intraperitoneal injection; enteral routes of administration e.g., administration by the oral route, lozenges, compressed tablets, pills, tablets, capsules, drops (e.g., ear drops), syrups, suspensions and emulsions; transdermal routes of administration; and inhalation (e.g., nasal sprays).

In some instances, compositions can be administered to a subject, e.g., a subject identified as being in need of treatment, using a systemic or local route of administration. Such local routes of administration include administering one or more agents into the ear of a subject and/or the inner ear of a subject, for example, by injection and/or using a pump.

In some instances, compositions can be can be injected into the ear (e.g., auricular administration), such as into the luminae of the cochlea (e.g., the Scala media, Sc vestibulae, and Sc tympani). For example, compositions can be administered by intratympanic injection (e.g., into the middle ear), intralabyrinthine delivery (e.g., to the stapes foot plate), and/or injections into the outer, middle, and/or inner ear. Such methods are routinely used in the art, for example, for the administration of steroids and antibiotics into human ears. Injection can be, for example, through the round window of the ear or through the cochlea capsule. In another exemplary mode of administration, compositions can be administered in situ, via a catheter or pump. A catheter or pump can, for example, direct a pharmaceutical composition into the cochlea luminae or the round window of the ear. Exemplary drug delivery apparatus and methods suitable for administering one or more compounds into an ear, e.g., a human ear, are described by McKenna et al., (U.S. Publication No. 2006/0030837) and Jacobsen et al., (U.S. Pat. No. 7,206,639). In some embodiments, a catheter or pump can be positioned, e.g., in the ear (e.g., the outer, middle, and/or inner ear) of a subject during a surgical procedure. In some embodiments, a catheter or pump can be positioned, e.g., in the ear (e.g., the outer, middle, and/or inner ear) of a subject without the need for a surgical procedure.

In some instances, the present disclosure includes treating a subject by administering to the subject cells produced using the agents and methods disclosed herein. In general, such methods can be used to promote complete or partial differentiation of a cell to or towards a mature cell type of the inner ear (e.g., a hair cell) in vitro. Cells resulting from such methods can then be transplanted or implanted into a subject in need of such treatment. Cell culture methods required to practice these methods, including methods for identifying and selecting suitable cell types, methods for promoting complete or partial differentiation of selected cells, methods for identifying complete or partially differentiated cell types, and methods for implanting complete or partially differentiated cells are described herein. Target cells suitable for use in these methods are described above.

Administration of cells to a subject, whether alone or in combination with agents disclosed herein, can include administration of undifferentiated, partially differentiated, and fully differentiated cells, including mixtures of undifferentiated, partially differentiated, and fully differentiated cells. As disclosed herein, less than fully differentiated cells can continue to differentiate into fully differentiated cells following administration to the subject.

Where appropriate, following treatment, the subject can be tested for an improvement in hearing or in other symptoms related to inner ear disorders. Methods for measuring hearing are well-known and include pure tone audiometry, air conduction, and bone conduction tests. These exams measure the limits of loudness (intensity) and pitch (frequency) that a subject can hear. Hearing tests in humans include behavioral observation audiometry (for infants to seven months), visual reinforcement orientation audiometry (for children 7 months to 3 years); play audiometry for children older than 3 years; and standard audiometric tests for older children and adults, e.g., whispered speech, pure tone audiometry; tuning fork tests; brain stem auditory evoked response (BAER) testing or auditory brain stem evoked potential (ABEP) testing. Oto-acoustic emission testing can be used to test the functioning of the cochlear hair cells, and electro-cochleography provides information about the functioning of the cochlea and the first part of the nerve pathway to the brain. In some embodiments, treatment can be continued with or without modification or can be stopped.

### EXAMPLES

In order that the invention described may be more fully understood, the following examples are set forth. The examples described in this application are offered to illustrate the methods and compositions provided herein and are not to be construed in any way as limiting their scope.

### Materials and Methods

The following materials and methods were used in the Examples set forth herein.

### Mouse Models

Rosa-rtTA (rtTA), Sox2Cre transgenic mice and Ai14 tdTomato reporter mice were from Jackson Laboratory (Stock #006965; 017593; 007914); tet-on-Myc mice were from Dr. M. Bishop of the University of California, San Francisco; tet-on-NICD mice were from Dr. D. Melton of Harvard University. For the transgenic rtTA/tet-on-Myc/tet-on-NICD mice, the background was mixed C57/129SvJ/CD1, with roughly equal numbers of sexes; Atoh1-nGFP mice were from Dr. Jane Johnson (University of Texas Southwestern Medical Center, Dallas, TX); the wild type mice were C57BL/6 from Jackson Laboratory. All experiments were performed in compliance with ethical regulations and approved by the Animal Care Committees of Massachusetts Eye and Ear and Harvard Medical School.

### Adult Cochlear Culture and Viral Infection In Vitro

Different from the neonatal cochlear culture method, in which the cochleae were disassociated from the bone, adult mouse whole cochleae (4-6 weeks old) were dissected with the bone attached. The bulla was first removed from the skull and dipped in 75% ethanol for 3 minutes before being placed in HBSS. The vestibular region was also removed. Under a dissecting microscope, the middle ear, vessels and the debris were removed from the bulla. The bone covering the apical turn was removed, and round window and oval window membranes were opened to allow media exchange with the cochlear fluids. The ligament portion and Reissner's membrane at each end of the cochlea were also removed to facilitate the access of medium to the sensory epithelial region. The cochleae were maintained in DMEM/F12 (Invitrogen) supplemented with N2 and B27 (both from Invitrogen) for 14-18 days. Ad-Atoh1 ad-Atoh1-mCherry adenoviruses were purchased from the SignaGen Laboratories, Rockville, MD, with titers of 6×10¹⁰ pfu/ml.

### Reprogramming In Vitro for HC Regeneration

In the rtTA/tet-Myc/tet-NICD mouse model, cochleae were treated with Dox (Sigma, 2 μg/ml final concentration); In the Sox2/tdT/rtTA/tet-Myc/tet-NICD mouse model, cochleae were treated with Dox (Sigma, 2 μg/ml final concentration) and 4-Hydroxytamoxifen (Tm, Sigma) (20 ng/ml); in the rtTA/tet-Myc and rtTA/tet-Myc/Atoh1-GFP mouse models, cochleae were treated with Dox (Sigma, 2 μg/ml final concentration) and VPA (2 mM); in the Sox2/tdT/rtTA/tet-Myc mouse model, cochleae were treated with 4-Hydroxytamoxifen (20 ng/ml), Dox (Sigma, 2 μg/ml) and VPA (Sigma, 2 mM); in the rtTA/tet-NICD mouse model, cochleae were treated with Dox (Sigma, 2 μg/ml final concentration), LiCl (Sigma, 8 mM), FSK (Tocris Bioscience, 20 μM), siFIR (Santa Cruz Bio-technology, 0.02 μM), and siMxi1 (Santa Cruz Bio-technology, 0.02 μM); in the Sox2/tdT/rtTA/tet-NICD mouse model, cochleae were treated with 4-Hydroxytamoxifen (20 ng/ml), Dox (Sigma, 2 μg/ml final concentration), LiCl (8 mM), Forskolin (FSK) (20 μM), siFIR (Puf 60, Gene ID: 67959) (0.02 M), and siMxi1 (Gene ID: 17859) (0.02 μM); in the Atoh1-GFP mouse model, cochleae were treated with VPA (2 mM), LiCl (8 mM), FSK (20 M), siFIR (0.02 μM), and siMxi1 (0.02 μM) for 4 days, followed by ad-Atoh1 (6×10¹⁰ pfu/ml) infection overnight. siRNAs were delivered following the manufacturer's instructions (Polyplus-transfection; 89129-920). In cultured adult mouse cochleae, they were treated with HDAC inhibitor, Belinostat (1 μM, Medchemexpress), Trichostatin A (TSA)(82.5 nM, Medchemexpress), Vorinostat (SAHA)(2 μM; Medchemexpress), and Myc siRNA Fbxw7 (0.02 μM, Santa Cruz Biotechnology). The controls were cultured adult cochleae of the same genotype treated with vehicle (sterile water with 0.1% DMSO) plus ad-Atoh1. The culture was placed into fresh medium for an additional 10-14 days. Cochleae were harvested and decalcified before immunohistochemistry.

Whole utricles were dissected from 4-week-old mice and cultured free-floating. Otoconia were removed using a gentle stream of phosphate-buffered saline (PBS) ejected from a 25G needle and syringe. Utricles were cultured in 1000 μL of media in untreated 24-well flat bottom plates. All cultures were maintained at 37° C. in 5% CO2/95% air.

Culture media consisted of Dulbecco's Modified Eagle's Medium/F12 (DMEM/F12, Invitrogen) with B27 and N2 supplement. Neomycin sulfate stock (10 mg/mL in 0.9% NaCl, from Sigma Aldrich) was diluted in culture media to 4 mM. Valproic acid (VPA) from Sigma was included at 2 mM, and siRNAs from Santa Cruz were included at 0.02 μM.

### Lineage Tracing

Cochlear tissues from 4- to 6-week-old Sox2-CreER/tdT mice, Sox2-CreER/tdT/rtTA/tet-MYC quad mice, Sox2-CreER/tdT/rtTA/tet-NICD quad mice, and Sox2-CreER/tdT/rtTA/tet-MYC/tet-NICD quint mice were dissected for culture. 4-Hydroxytamoxifen (20 ng/mL) was added to cultures on day 0 to activate Cre for lineage tracing studies. Ad-Atoh1-V5 virus was added to the medium for 16 to 24 hours at a concentration of 6×10¹⁰ pfu/ml.

Trans-Tympanic Injection of Chemicals and siRNA In Vivo

All adult mice used were between 4 and 6 weeks old. Trans-tympanic injections were performed 7 days after the subcutaneous injection of Kanamycin (0.8 mg/g; Sigma) followed by intraperitoneal injection of Furosemide (0.3 mg/g; Hospira Inc) 30 min later. Mice were anesthetized by intraperitoneal injection of xylazine (10 mg/kg) and ketamine (100 mg/kg). Trans-tympanic injections were conducted with 5 μl chemical combinations of VPA (5 mg/ml), LiCl (40 mM), FSK (50 g/ml), siFIR (0.6 g/10 l), and siMxi1 (0.6 g/10 l), or vehicle (ddH₂O with 0.5% DMSO). Chemicals or vehicle were injected into one ear through the tympanic membrane (TM) in mice. Microforceps were used to retract the skin and visualize the medial superior fold adjacent to the TM. A Hamilton syringe with 33G needle was used to inject drugs trans-tympanically.

### Viral Injection In Vivo

All surgical procedures were done in a clean, dedicated space. Instruments were thoroughly cleaned with 70% ethanol and autoclaved before surgery. Mice were anesthetized by intraperitoneal injection of xylazine (10 mg/kg) and ketamine (100 mg/kg). For viral injection, cochleostomy was performed on the anesthetized mice by opening the bulla, and adenovirus with a titer of 5×10¹² pfu/ml was injected into the middle turn of the scala media by a pressure-controlled motorized microinjector at a speed of 3 nl/sec. A total of 1 μl of adenovirus was injected into each cochlea.

### Example 1: Novel Combinations of siRNA and Chemical Compounds for Hair Cell Regeneration

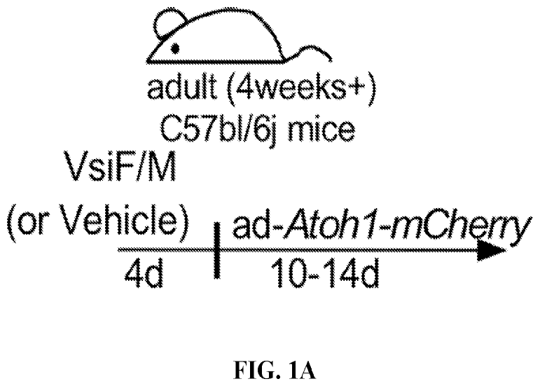

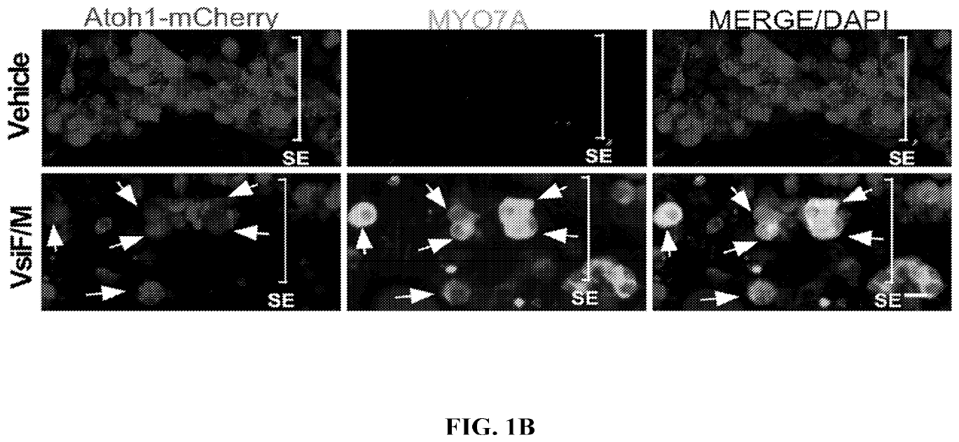

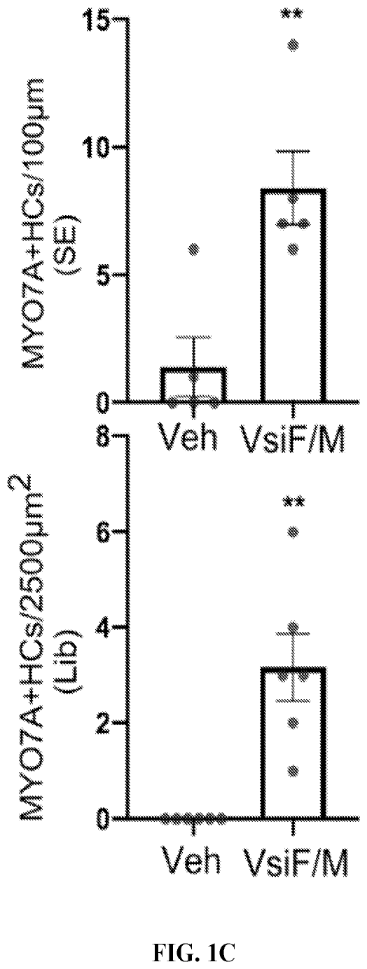

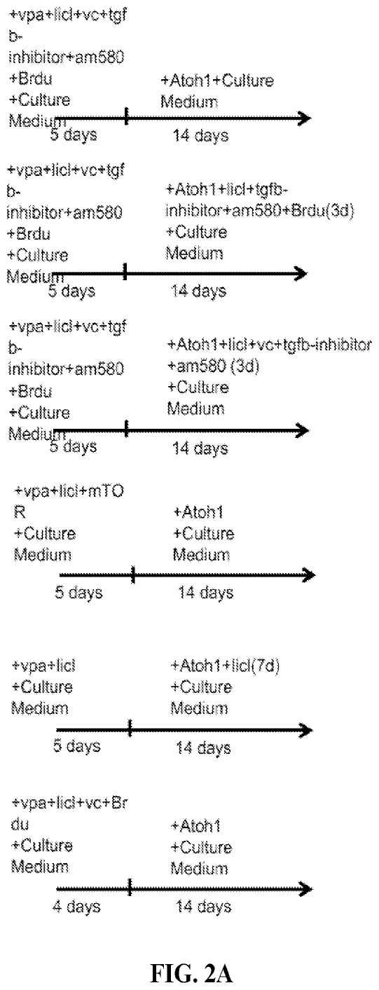

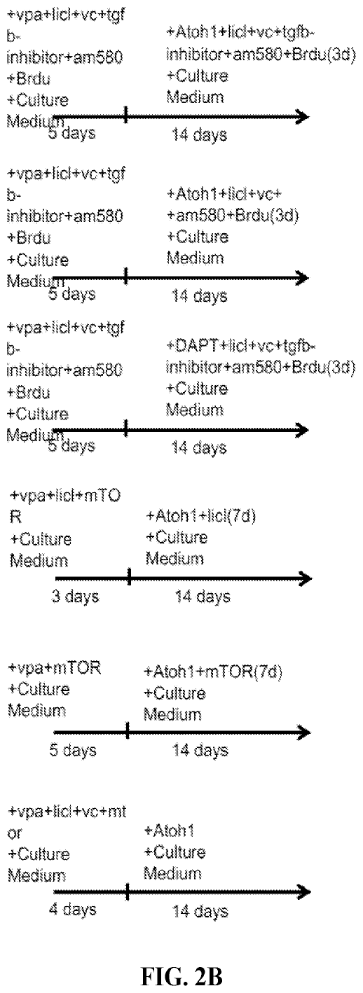

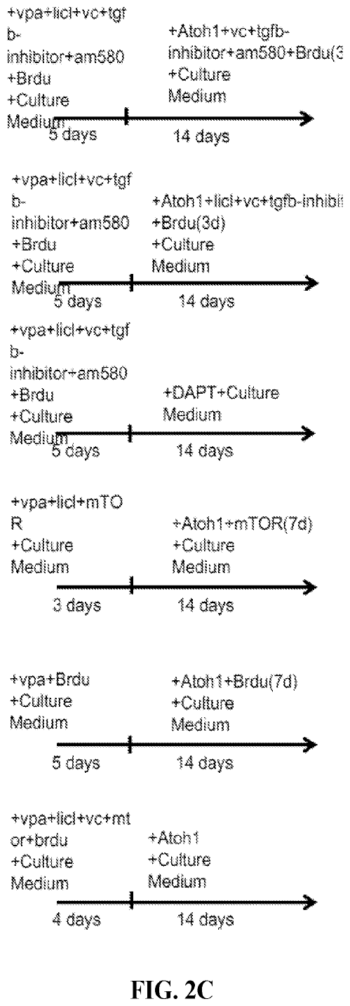

Various combinations of small molecules and siRNAs were screened for reprogramming and regenerating hair cells in cultured adult mouse cochlea. The small molecules and siRNAs included in the screen targeted various pathways including Notch, Myc, mTOR, Wnt, Tgfb, FGF, retinoic acid, BMP4, and Alk5 pathways. Methyltransferase inhibitors and HDAC inhibitors were also screened for the ability to reprogram and regenerate hair cells in cultured adult mouse cochlea. Of the various combinations tested, the combination of Valproic acid (VPA) with two siRNAs (siFIR (siF), and siMxi1 (siM)) enhanced cochlear reprogramming. As shown in FIGS. 1A-1C, new MYO7A+/Atoh1-mCherry+HCs were detected in the adult wild type mice cochleae in vitro after reprogramming with combination of VPA and siF/M followed by the addition of ad-Atoh1-mCherry. Other combinations failed to achieve hair cell regeneration (FIGS. 2A-2C).

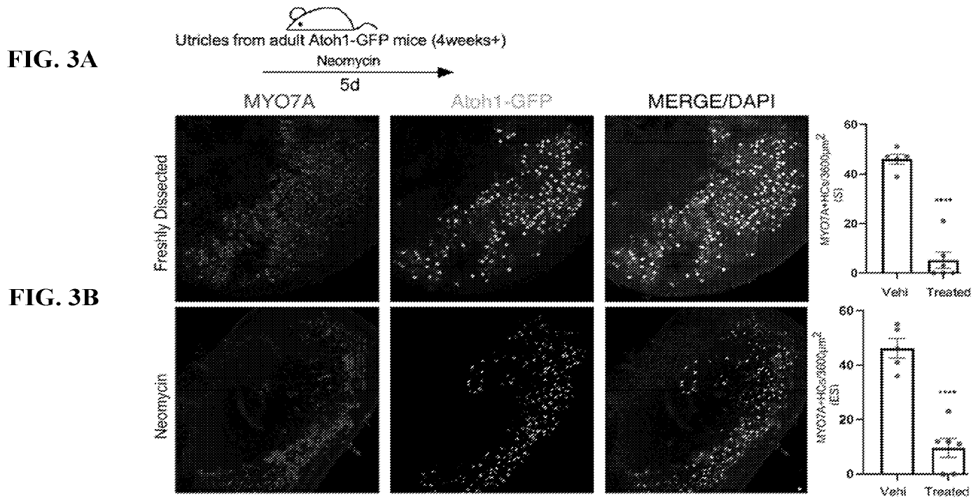

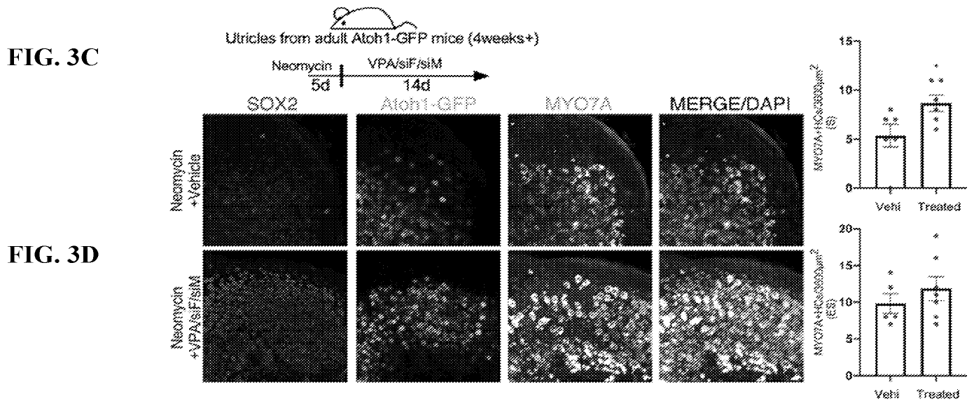

Next, we tested whether treatment with the novel “VLFsiFsiM cocktail” could regenerate HCs in the utricle. After treatment with neomycin for 5 days, dissected utricles had decreased total numbers of utricle HC compared to untreated dissected utricles (FIGS. 3A-3B). After the treatment with neomycin, cultured adult utricles were treated with the VPA/siF/siM cocktail for 14 days. Treated utricles showed an increase in the number of hair cells (MYO7A+/Atoh1-GFP+) compared to the vehicle (DMSO) treated group (FIGS. 3C-3D). Notice that in the utricle, hair cells can be regenerated by the “VLFsiFsiM cocktail” alone without Atoh1.

Taken together, we demonstrated that the treatment of the novel “VLFsiFsiM cocktail” is sufficient to reprogram the adult mouse cochlea and utricle for HC regeneration with or without Atoh1.

### Example 2: Regeneration of Hair Cells in Wild-Type Mice

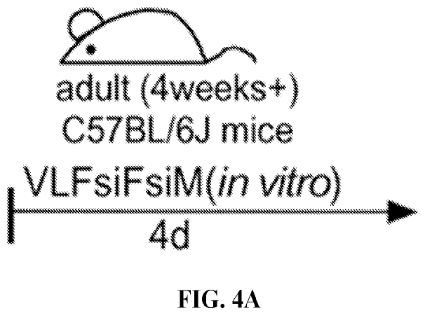

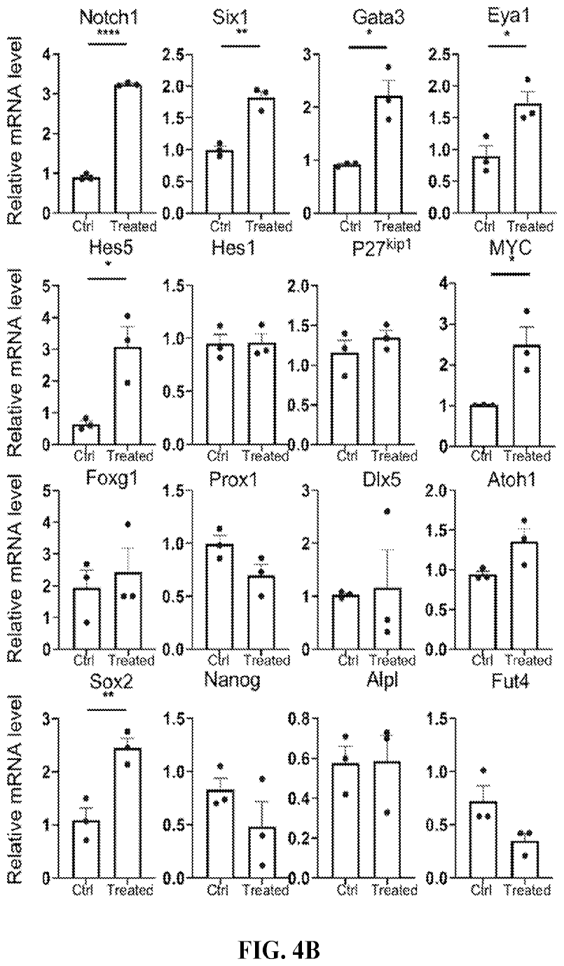

To explore whether the Wnt and cAMP pathways are sufficient to reprogram the adult cochlea, we combined the previous chemicals VPA, siFIR, and siMxi1 (VsiFsiM) with a Wnt agonist (LiCl, L) and a cAMP agonist Forsklin (FSK, F) to make a “cocktail” with 5 chemicals (VLFsiFsiM). Treatment with the cocktail triggered adult wild type mice cochleae to re-express inner ear progenitor genes including Six1, Eya1 and Gata3, but not embryonic stem cell genes like Nanog and Fut4 (FIGS. 4A-4B).

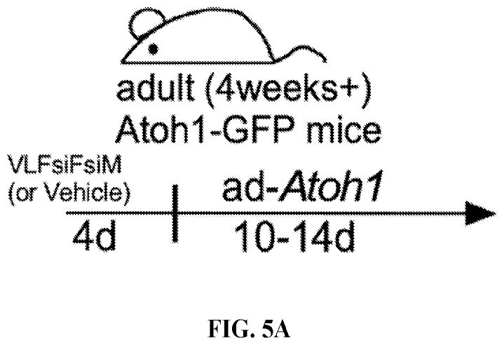

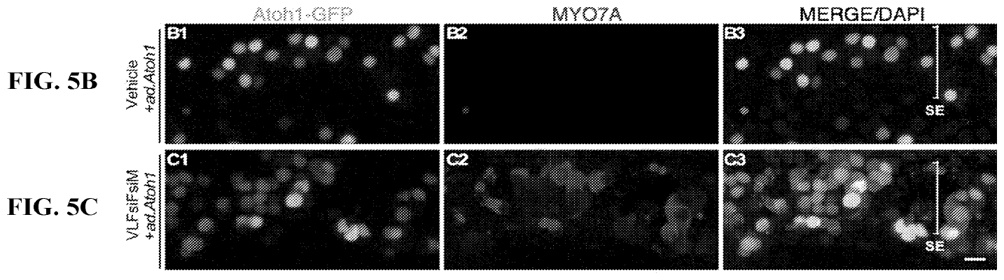

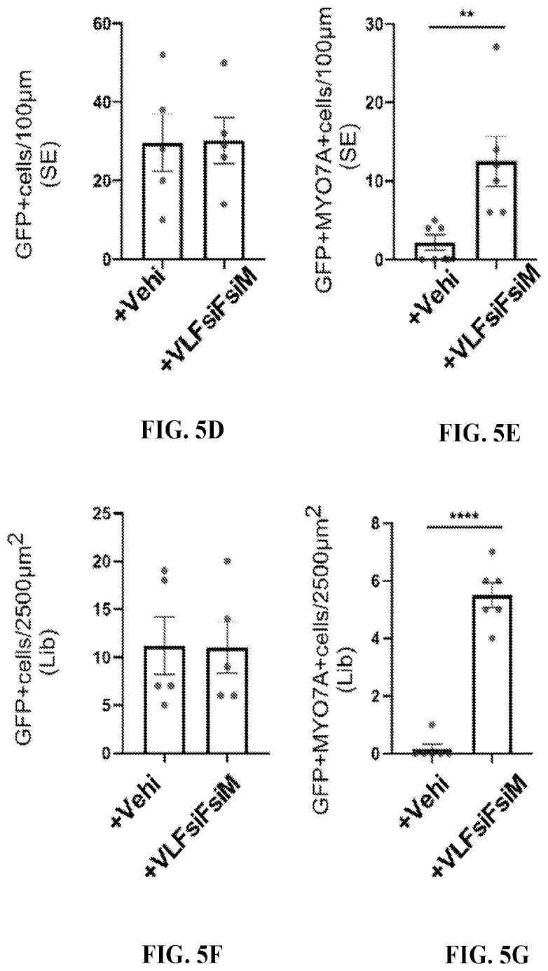

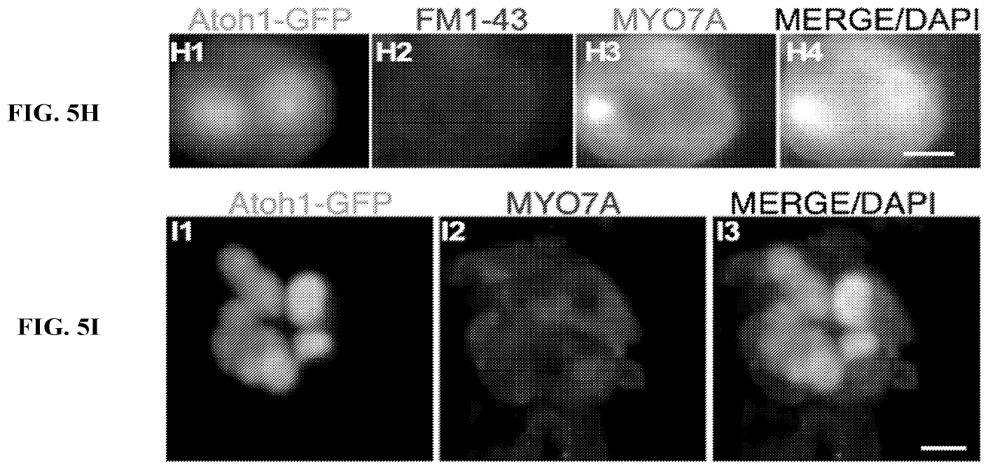

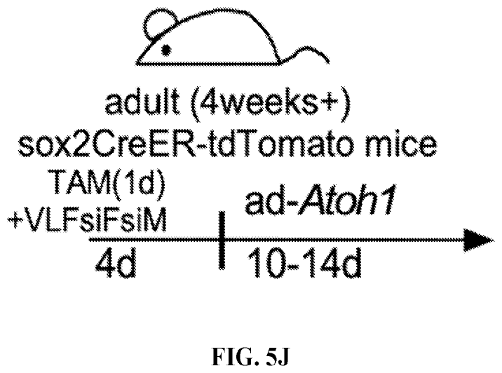

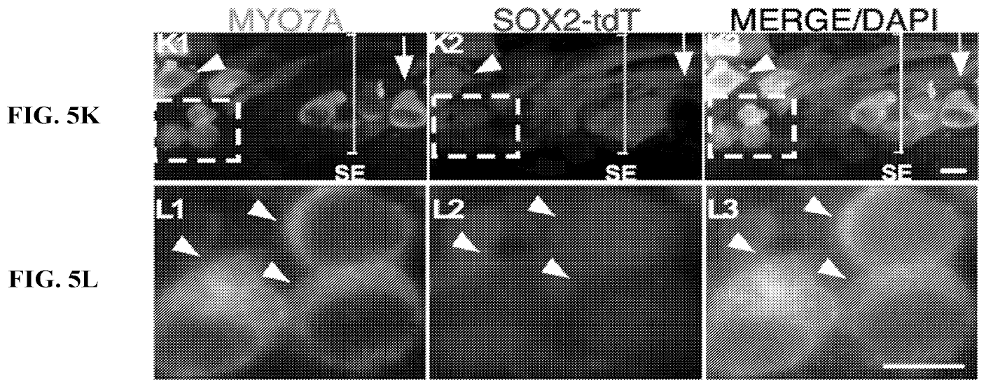

To further trace the source of HC regeneration potential, we added the VLFsiFsiM cocktail to reprogram the adult Atoh1-GFP mouse cochleae, in which the Atoh1 enhancer drives expression of a nuclear GFP reporter in the Atoh1 lineage (FIG. 5A). Followed by ad.Atoh1 induction, we found a large number of Atoh1-GFP+/MYO7A+HCs regenerated in the adult cochleae in vitro (FIGS. 5B-5G). In contrast, in control cochlea without cocktail treatment, virtually no Atoh1-GFP+/MYO7A+HCs were regenerated (FIGS. 5B-5G). We performed the FM1-43 uptake experiment to study if the new HCs possess functional transduction channel. This fluorescent dye pass through functional transduction channels and are trapped by their charge with HCs. Notably, FM1-43+/Atoh1-GFP+/MYO7A+ triple positive HCs were generated in the cocktail treated group, indicating the up-take of the dye by the HCs (FIG. 511). Interestingly, we found Atoh1-GFP+/MYO7A+ sphere-like structures in the cocktail treated group (FIG. 5I), indicating a progenitor/stem cell signal might be turned on by reprogramming with the cocktail. We performed linage tracing study using the Sox2CreER-tdTomato (tdT) mice in which Tamoxifen (TAM) treatment permanently labels supporting cells with tdT. After the cocktail treatment and ad-Atoh1 infection, new regenerated hair cells (MYO7A+) were found to be tdT positive, which demonstrates that they were derived from transdifferentiation of SOX2+ supporting cells (FIGS. 5J-5L).

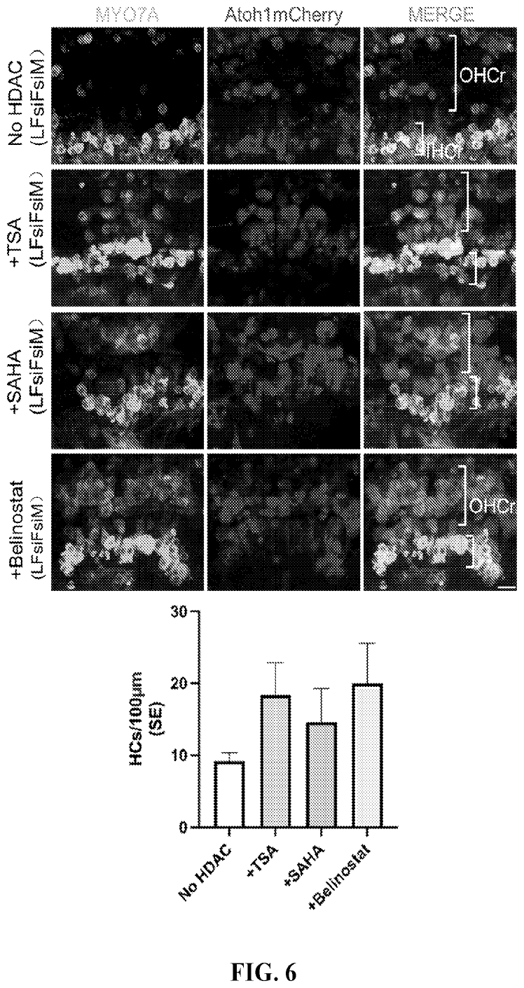

Next, we tested whether HDAC inhibitors other than VPA could be used to reprogram adult wild-type cochlea for hair cell regeneration. The experiments described above were performed using the HDAC inhibitors TSA (82.5 nM), SAHA (2 μM), or Belinostat (1 μM) in the cocktail instead of VPA. Interestingly, each of the HDAC inhibitors was sufficient to induce reprogramming and efficient regeneration of hair cells in adult wild-type cochlea after Ad-Atoh1 infection (FIG. 6).

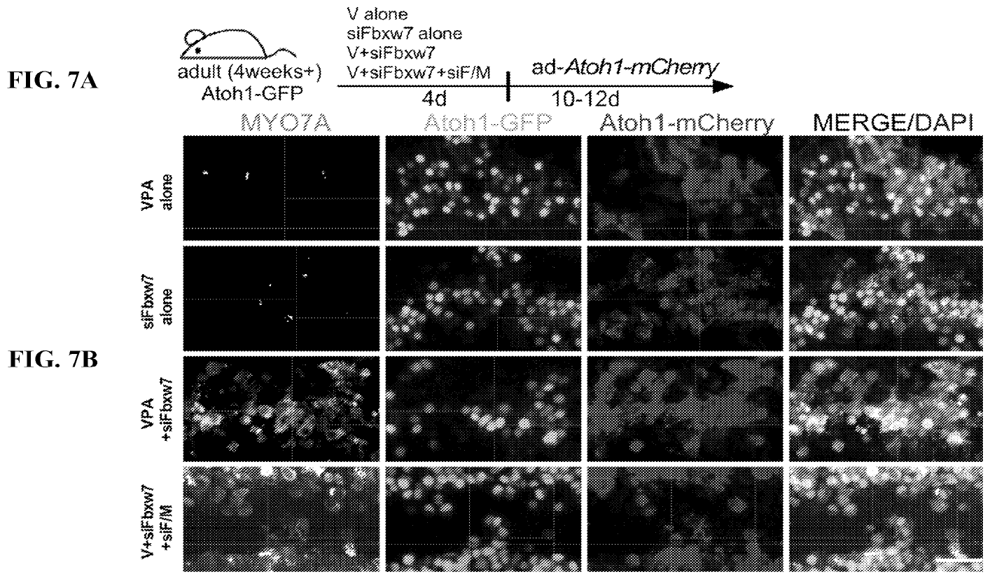

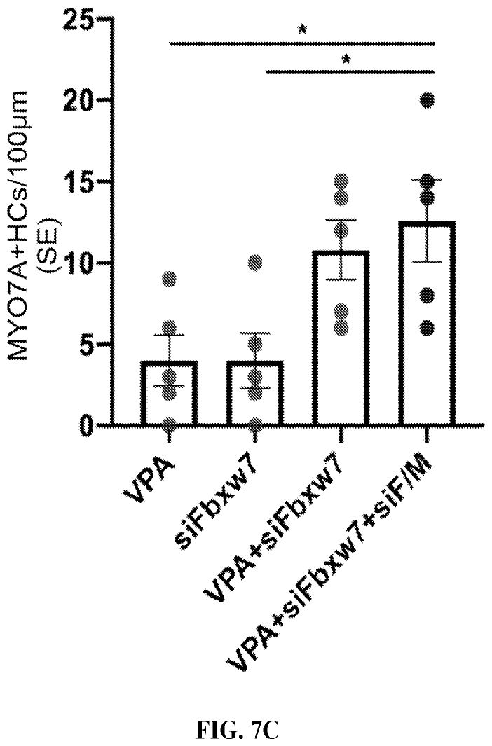

To test whether siRNA targeting the Myc modulator Fbxw7 could be used to promote hair cell regeneration, adult wild-type mouse cochlea were treated with siRNA targeting Fbxw7 (siFbxw7) alone or in combination with VPA. Experiments were also performed using siFbxw7 in combination with siF and siM (FIG. 7A). In cultured adult WT cochlea, VPA treatment and Ad-Atoh1 infection did not lead to HC (MYO7A) (FIG. 7B). Fbxw7 siRNA treatment alone and Ad-Atoh1 infection did not regenerate hair cells (MYO7A+) (FIG. 7B). By contrast, treatment with the HDAC inhibitor VPA and siFbxw7 alone or in combination with siF and siM followed by Ad-Atoh1 infection induced HC (MYO7A+) regeneration in cultured adult WT cochlea (FIG. 7B). Quantification showed increased hair cells as the result of regeneration under the different tested conditions (FIG. 7C).

Taken together, we demonstrated that the treatment with the novel “VLFsiFsiM cocktail” is sufficient to reprogram the adult mouse cochlea for HC regeneration with Atoh1. We also demonstrated that various HDAC inhibitors (VPA, TSA, SAHA, or Belinostat) could be used in combination with siRNAs targeting the Myc modulators Fir, Mix1, or Fbxw7 to regenerate HCs.

### Example 3: Robust Regeneration of HCs in the Severe HC Loss Mouse Model

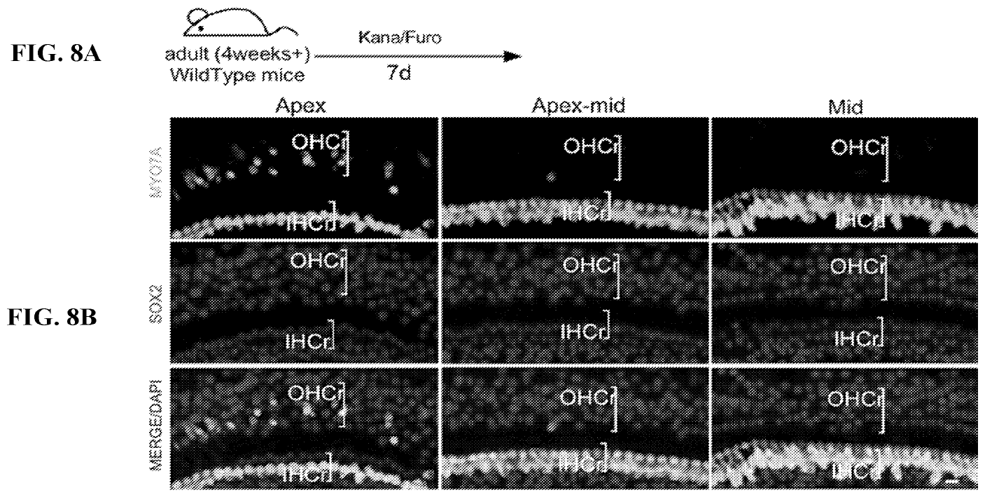

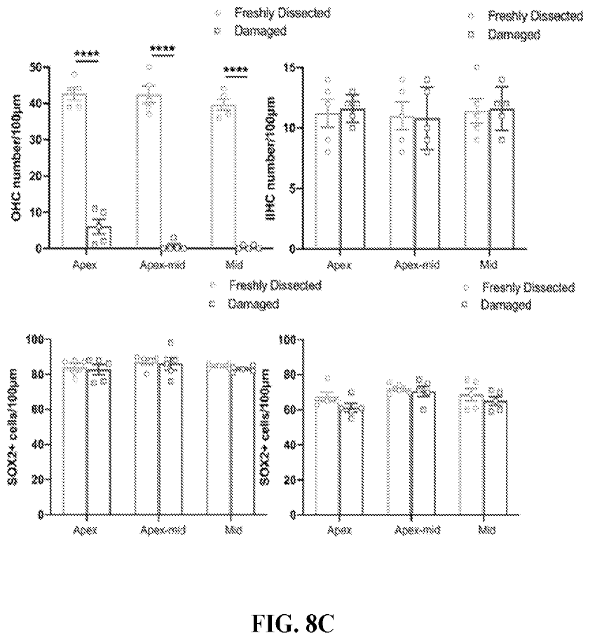

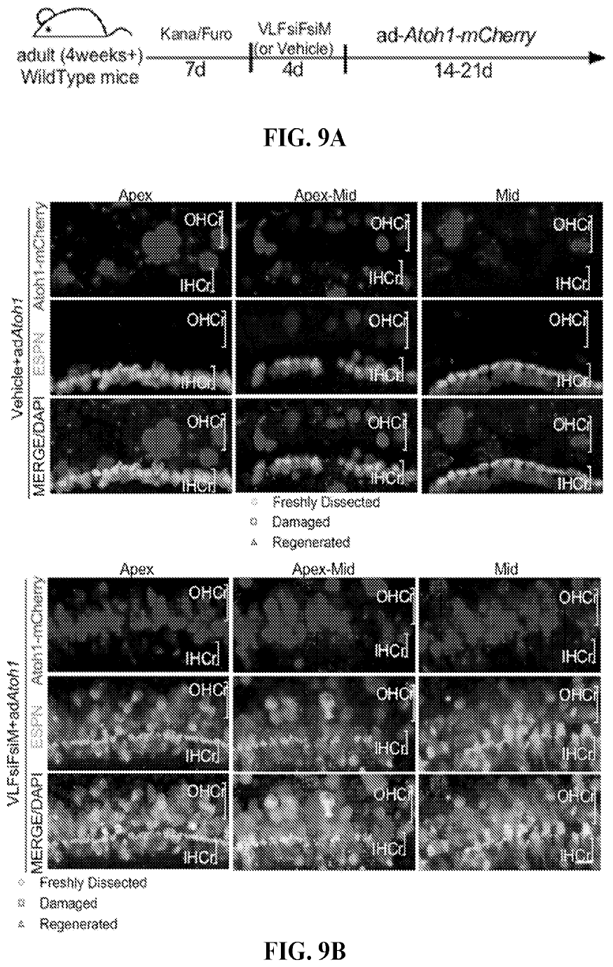

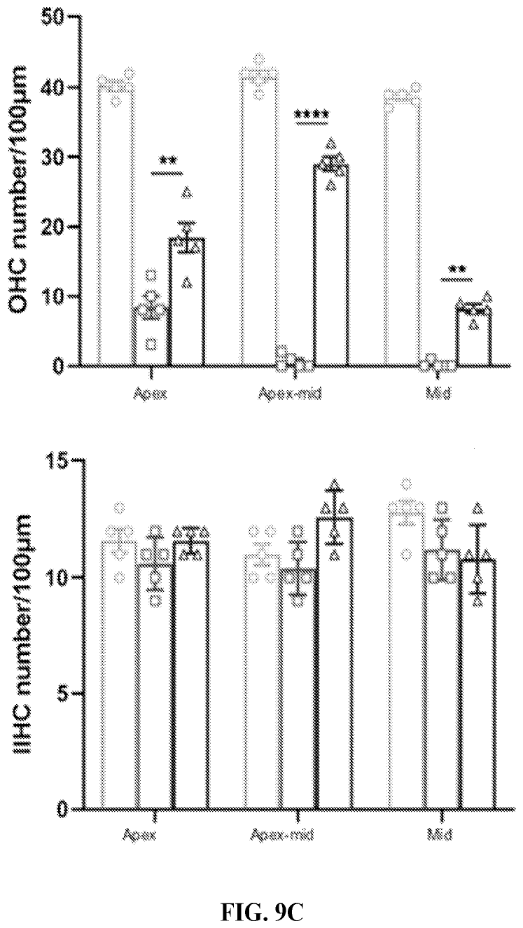

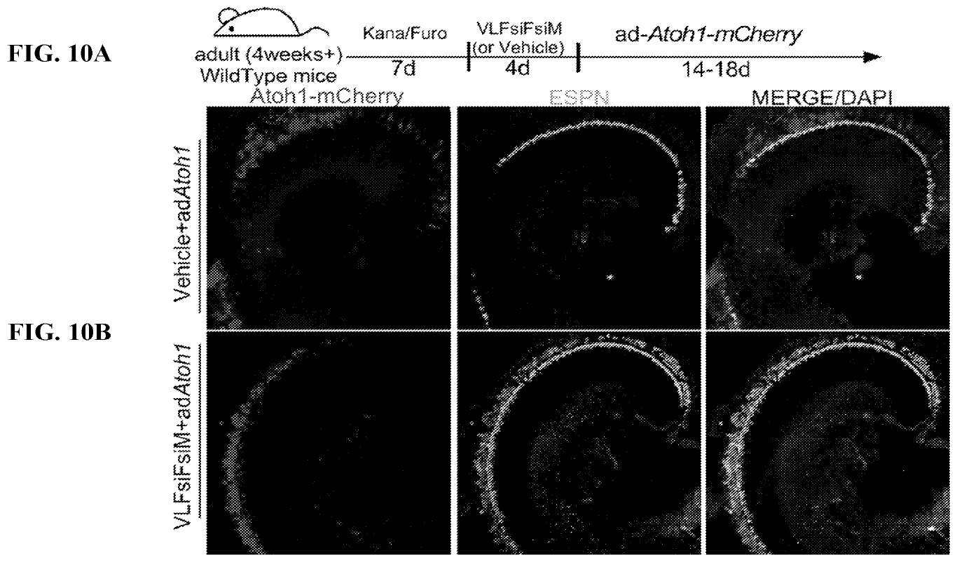

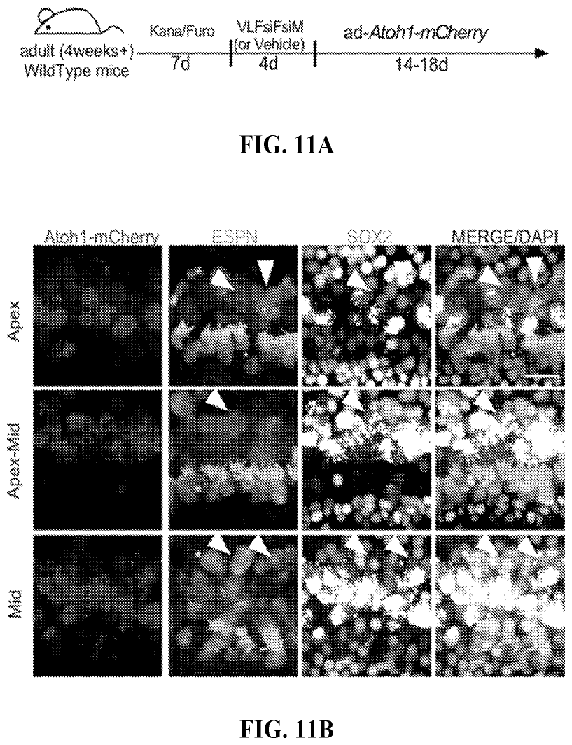

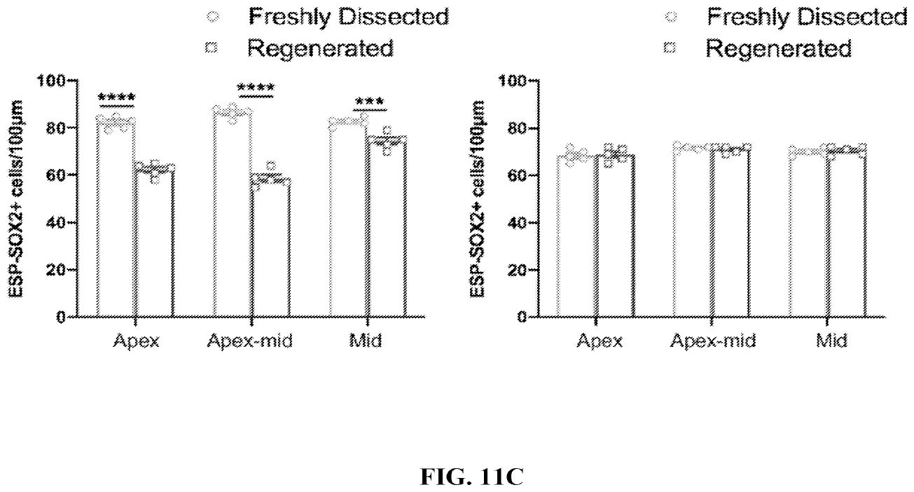

To further explore our HC regeneration protocol for future clinical applications, we tested our protocol in a severe HC loss mouse model. We observed that over 95% of OHCs disappeared 7 days after intraperitoneal (i.p.) injection of Kanamycin and Furosemide. Remarkably, nearly all OHCs were wiped out in the Apex-mid and Mid turn, while almost all SOX2+SCs were preserved, making it a suitable model for detecting SC—HC trans-differentiation in vivo (FIGS. 8A-8C). After the severe loss of the OHCs, we treated the damaged cochleae by injecting the VLFsiFsiM cocktail, then regenerated HCs by Ad.Atoh1mCherry infection via cochleostomy at the middle turn of the cochlea. 14-21 days later, multiple Atoh1mCherry+ cells were detected in the SE region throughout the Apex to the Mid turn of the cochlea, most of which were in the OHC region. Significantly more ESPN+ cells in the OHC region were observed throughout the cocktail-reprogrammed cochleae compare to the Vehicle-treated un-reprogrammed cochleae (FIGS. 9A-9C and FIGS. 10A-10B). Notably, many new regenerated HCs were SOX2+/ESPN+HCs, reflecting their newly regenerated status (FIGS. 11A-11B). Consistent with that, the total number of non-transdifferentiated ESPN−/SOX2+SCs was significantly reduced in the reprogramed cochleae, indicating the trans-differentiation of SCs to HCs (FIG. 11C). A robust regeneration of HCs in the OHC region was observed by video (data not shown).

Taken together, we demonstrated that the treatment with the novel “VLFsiFsiM cocktail” is sufficient to reprogram the adult mouse cochlea and regenerate HCs in combination with Atoh1 in a severe HC loss mouse model in vivo.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for reprogramming an adult mammalian inner ear for hair cell regeneration, the method comprising:
contacting an adult mammalian inner ear with an effective amount of a histone deacetylase (HDAC) inhibitor and one or more inhibitory nucleic acids targeting Fir, Mxi1, Fbxw7, or a combination thereof, under conditions and for a time sufficient to produce a population of progenitor cells in the adult mammalian inner ear.

2. The method of claim 1, wherein the HDAC inhibitor is selected from the group consisting of Sodium Butyrate, Trichostatin A, hydroxamic acids, cyclic tetrapeptides, trapoxin B, depsipeptides, benzamides, electrophilic ketones, aliphatic acid compounds, pyroxamide, phenylbutyrate, valproic acid, hydroxamic acids, romidepsin, vorinostat (SAHA), belinostat (PXD101), LAQ824, panobinostat (LBH589), entinostat (MS275), CI-994 (N-acetyldinaline, also tacedinaline), Entinostat (SNDX-275; formerly MS-275), EVP-0334, SRT501, CUDC-101, JNJ-26481585, PCI24781, Givinostat (ITF2357), and mocetinostat (MGCD0103).

3. The method of claim 2, wherein the HDAC inhibitor is valproic acid, Trichosatin A, vorinostate (SAHA), or belinostat (PXD101).

4. The method of claim 1, wherein the one or more inhibitory nucleic acids is a small interfering RNA (siRNA), a short hairpin RNA (shRNA), or an antisense oligonucleotide.

5. The method of claim 1, wherein the one or more inhibitory nucleic acids comprises inhibitory nucleic acids that target Fir and Mxi1.

6. The method of claim 1, further comprising contacting the mammalian cochlea with a Wnt agonist and/or a cAMP agonist.

7. The method of claim 6, wherein the Wnt activator is lithium chloride (LiCl) and/or the cAMP activator is forskolin.

8. The method of claim 1, wherein the progenitor cells of the population express Six1, Eya1, Gata3, Sox2, Notch1, Hes5, or a combination thereof.

9. The method of claim 1, wherein the contacting occurs in the inner ear of a subject.

10. A method for treating hearing loss or vestibular dysfunction in a subject, the method comprising:
administering to an inner ear of a subject in need thereof an effective amount of a histone deacetylase (HDAC) inhibitor and one or more inhibitory nucleic acids targeting Fir, Mxi1, Fbxw7, or a combination thereof, and
administering to the inner ear of the subject an effective amount of an Atoh1 activator.

11. The method of claim 10, wherein the HDAC inhibitor is selected from the group consisting of Sodium Butyrate, Trichostatin A, hydroxamic acids, cyclic tetrapeptides, trapoxin B, depsipeptides, benzamides, electrophilic ketones, aliphatic acid compounds, pyroxamide, phenylbutyrate, valproic acid, hydroxamic acids, romidepsin, vorinostat (SAHA), belinostat (PXD101), LAQ824, panobinostat (LBH589), entinostat (MS275), CI-994 (N-acetyldinaline, also tacedinaline), Entinostat (SNDX-275; formerly MS-275), EVP-0334, SRT501, CUDC-101, JNJ-26481585, PCI24781, Givinostat (ITF2357), and mocetinostat (MGCD0103).

12. The method of claim 11, wherein the HDAC inhibitor is valproic acid, Trichosatin A, vorinostate (SAHA), or belinostat (PXD101).

13. The method of claim 10, wherein the one or more inhibitory nucleic acids is a small interfering RNA (siRNA), a short hairpin RNA (shRNA), or an antisense oligonucleotide.

14. The method of claim 10, wherein the one or more inhibitory nucleic acids targets Fir and Mxi1.

15. The method of claim 10, wherein the Atoh1 activator is a nucleic acid encoding Atoh1.

16. The method of claim 15, wherein the nucleic acid encoding Atoh1 is comprised in a vector.

17. The method of claim 16, wherein the vector is a viral vector.

18. The method of claim 17, wherein the viral vector is selected from the group consisting of a retroviral vector, a lentiviral vector, an adenoviral vector, an adeno-associated viral vector, a herpes simplex viral vector, and a vaccinia viral vector.

19. The method of claim 10, further comprising administering to the subject a Wnt agonist and/or a cAMP agonist.

20. The method of claim 19, wherein the Wnt activator is lithium chloride (LiCl) and/or the cAMP activator is forskolin.

21. The method of claim 10, wherein the subject is a human patient having noise-induced permanent deafness, drug-induced hearing loss, age-related hearing loss, sudden sensorineural hearing loss, hearing loss due to viral infection, tinnitus, vestibular dysfunction, or a combination thereof.

22. The method of claim 10, wherein the subject is a human.
